(19) United States
(12) Patent Application Publication
(10) Pub. No.: US 2010/0022919 A1
(43) Pub. Date: Jan. 28, 2010

(54) Methods of Skin Grafting Using Ultrasound

(75) Inventor: Michael T. Peterson, Lakeville, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Celleration, Inc., Eden Prairie, MN (US)

(21) Appl. No.: 12/507,289

(22) Filed: Jul. 22, 2009

Related U.S. Application Data
(60) Provisional application No. 61/135,741, filed on Jul. 22, 2008.

(51) Int. Cl. A61N 7/00

(52) U.S. Cl. 601/2; 604/22

(57) ABSTRACT

The present invention provides improved methods for skin grafting using ultrasonic energy.

### RELATED APPLICATIONS

This application claims the benefit of priority to U.S. provisional application No. 61/135,741, filed Jul. 22, 2008. The disclosure of the foregoing application is incorporated by reference in its entirety.

### BACKGROUND

Ultrasound waves have been widely used in medical applications. For example, ultrasound waves have been used for diagnostic and therapeutic purposes, as well as in many industrial applications. One diagnostic use of ultrasound waves includes using ultrasonic waves to detect underlying structures in an object or a human tissue. In this procedure, an ultrasonic transducer is placed in contact with the object or tissue via a coupling medium and high frequency (1-10 MHz) ultrasonic waves are directed into the tissue. Upon contact with various underlying structures, the waves are reflected back to a receiver adjacent the transducer. By comparison of the signals of the ultrasonic wave as sent with the reflected ultrasonic wave as received, an image of the underlying structure can be produced. This technique is particularly useful for identifying boundaries between components of tissue and can be used to detect irregular masses, tumors, and the like.

In addition to diagnostic uses, ultrasonic energy can also be used for therapeutic purposes. For example, in contact physiotherapy, ultrasonic energy is delivered to a tissue via direct contact between the device and the patient tissue. In conventional ultrasound physiotherapy, an ultrasonic wave contacts the tissue via a coupling medium. This direct contact, even if via a coupling medium, may be undesirable for certain medical applications, such as in the treatment of open wounds resulting from, for example, trauma, burns, and surgical interventions.

Commonly-owned U.S. Pat. No. 6,569,099, and application Ser. Nos. 11/473,934, 10/409,272, 10/815,384, and 12/006,739 disclose ultrasonic systems, devices and methods for wound treatment. The entire contents of each of the foregoing patents and patent application are incorporated herein by reference. Briefly, these patents and applications disclose devices, systems, and methods for delivering ultrasound energy, in the presence or absence of a liquid spray, to a wound via an applicator. The ultrasound energy, and when present a liquid spray, is delivered from a non-contact distance. Commonly-owned U.S. patent application Ser. Nos. 11/473,934 and 12/006,739, the entire contents of which are incorporated herein by reference, additionally provide several examples of removable applicator nozzles that can be used with an ultrasound therapy device. The disclosed devices and systems can be used in non-contact methods for delivering ultrasonic energy to have a therapeutic effect.

Despite advances in wound care, some wounds require a skin graft to achieve complete wound closure. Often, the best source of tissue for a skin graft is skin taken from another region of the same patient's body. This region from which the skin graft is harvested is often referred to as the donor site.

Although obtaining an autograft has numerous benefits, the harvesting of tissue from the donor site may cause additional pain, as well as create another site where proper wound healing and infection prevention must be managed. As such, there is a need in the art for improved methods of managing skin grafting at a skin graft donor site and/or a skin graft recipient site.

### SUMMARY OF THE INVENTION

The present invention provides methods for managing skin grafting at a skin graft donor site and/or a skin graft recipient site. The present invention provides improved methods for performing skin grafting by delivering low frequency ultrasonic energy from a non-contact distance to a donor site and/or a recipient site. Delivering low frequency ultrasonic energy prior to, during and/or subsequent to graft harvest (when referring to the donor site) and/or graft placement (when referring to the recipient site) has numerous therapeutic benefits. By way of example, providing an effective amount of ultrasonic energy (in the presence or absence of a liquid mist) has therapeutic effects including one or more of the following: kill bacteria, decrease bacterial burden, decrease the presence of a biofilm, reduce pain, decrease inflammation, promote wound healing, promote epithelial cell proliferation and/or differentiation, prevent or reduce contraction, and prevent or reduce scarring. The therapeutic benefits of delivering ultrasonic energy include therapeutic benefits at the skin surface and/or at a depth beneath the skin surface.

In a first aspect, the invention provides a method of reducing pain associated with removal of a skin graft from a skin graft donor site. The method comprises providing a transducer which can emit low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site following removal of a skin graft from said site. The ultrasonic energy is delivered from a non-contact distance between a vibrating tip of the transducer and the skin graft donor site. The delivered ultrasonic energy provides a therapeutic effect to reduce pain associated with removal of a skin graft from said skin graft donor site.

In certain embodiments, the method further comprises at least one treatment with ultrasonic energy prior to removal of the skin graft from the donor site.

Without being bound by theory, the emitted ultrasonic energy may provide a therapeutic effect via action at the tissue surface and/or by penetrating the treated patient tissue to provide a therapeutic effect beneath the tissue surface. In certain embodiments, the emitted ultrasonic energy provides a therapeutic effect at the tissue surface and/or beneath the tissue surface.

In certain embodiments, the ultrasonic energy is delivered to the skin graft donor site and to a periwound area adjacent to said skin graft donor site. In certain embodiments, the ultrasonic energy is delivered for a time proportional to the area of the skin graft donor site. In certain embodiments, the ultrasonic energy is delivered for a time proportional to the area of the skin graft donor site plus the periwound area.

In certain embodiments, the skin graft is a full-thickness skin graft. In certain embodiments, the skin graft is a partial-thickness skin graft. In certain embodiments, the skin graft is an autograft. In certain embodiments, the skin graft is an allograft.

In certain embodiments, the ultrasonic energy is delivered via a liquid spray, and the method comprises delivering the low frequency ultrasonic energy and the liquid spray to said skin graft donor site. In certain embodiments, the liquid spray is generated by delivering liquid to a distal portion of the transducer. In certain embodiments, the ultrasonic energy is delivered in the absence of a liquid spray or coupling agent. Regardless of whether the ultrasonic energy is delivered “wet” or “dry”, in certain embodiments, a topical medicament is applied to the donor site prior to or following delivery of the ultrasonic energy.

In certain embodiments, the method is part of a therapeutic regimen combining one or more additional treatment modalities. In certain embodiments, the one or more additional treatment modalities comprises applying a topical medicament to the skin graft donor site prior to and/or following delivering said ultrasonic energy to said skin graft donor site.

In certain embodiments, the method comprises delivering ultrasonic energy at least twice per week for at least two weeks. In certain embodiments, the method comprises delivering ultrasonic energy at least three times per week for at least two weeks. In certain embodiments, the method comprises a single treatment with ultrasonic energy.

In certain embodiments, assessing a reduction in pain in a human patient is based on that patient's self assessment of their pain. The self assessment can then be compared to, for example, the same patient's self assessment prior to onset of ultrasound therapy or average pain self-assessment data gathered from similar patient populations undergoing similar skin graft harvest procedures in the absence of treatment with non-contact, low frequency ultrasound therapy. In certain other embodiments, assessing a reduction in pain in a human patient is based on evaluating the frequency and/or quantity of requested or required pain medication in comparison to the average pain management requirements of similar patient populations undergoing similar skin graft harvest procedures in the absence of treatment with non-contact, low frequency ultrasound therapy.

In a second aspect, the invention provides a method of preparing tissue for skin grafting. The method comprises providing a transducer which emits low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site and/or a skin graft recipient site, and the ultrasonic energy is delivered from a non-contact distance between a vibrating tip of the transducer and treated patient tissue to provide a therapeutic effect.

Without being bound by theory, the emitted ultrasonic energy may provide a therapeutic effect via action at the tissue surface and/or by penetrating the treated patient tissue to provide a therapeutic effect beneath the tissue surface. In certain embodiments, the emitted ultrasonic energy provides a therapeutic effect at the tissue surface and/or beneath the tissue surface.

In certain embodiments, the method comprises delivering ultrasonic energy to a skin graft donor site. In certain embodiments, the ultrasonic energy is delivered prior to removing a skin graft from the donor site. In other embodiments, the ultrasonic energy is delivered following removing a skin graft from the donor site. In still other embodiments, the ultrasonic energy is delivered over any one or more of the following multiple different time periods: both prior to and following removing a skin graft from the donor site; multiple separate treatments prior to removing a skin graft from the donor site; multiple separate treatments following removing a skin graft from the donor site; multiple treatments prior to and/or following removing a skin graft from the donor site. Optionally, ultrasonic energy may also be delivered to the recipient site prior to and/or following graft transplantation. Multiple treatments at the recipient site and/or donor site, as well as single treatments at the recipient site and/or donor site are contemplated.

In certain embodiments, following graft transplantation to the recipient site, treatment with ultrasonic energy is delayed (or suspended if used prior to transplantation) for approximately five, six, seven, eight, nine, ten days, or even about two weeks to allow the graft to “take” prior to beginning or resuming ultrasonic energy treatment.

In certain embodiments, the ultrasonic energy is delivered via a liquid spray. Delivery of ultrasonic energy via a liquid spray is sometimes referred to herein as “wet” delivery. When used “wet”, ultrasonic energy and the liquid spray are delivered to the treated tissue from a non-contact distance (e.g., without direct contact between the device used to deliver the ultrasonic energy and the treated patient tissue). By way of example, the liquid spray can be generated by delivering liquid to a distal portion of the transducer, for example to a portion of the transducer tip.

In certain embodiments, the ultrasonic energy is delivered in the absence of a liquid spray or coupling agent. Delivery of ultrasonic energy in the absence of a liquid spray or coupling agent is sometimes referred to herein as “dry” delivery. As with “wet” delivery, the ultrasonic energy is delivered from a non-contact distance.

In certain embodiments, the method further comprises harvesting a skin graft from the donor site and transplanting the skin graft to a recipient site. Exemplary skin grafts include, but are not limited to, full-thickness skin grafts and partial-thickness skin grafts. These methods may be used regardless of whether the skin graft is an autograft or an allograft. Accordingly, the invention contemplates embodiments in which the donor site and the recipient site are on the same patient, as well as embodiments in which the donor site and recipient site are on different patients. In certain embodiments, although the graft is an allograft, the donor is the recipient's identical twin.

In certain embodiments, the method is part of a therapeutic regimen combining one or more additional treatment modalities. Exemplary additional modalities include, but are not limited to, negative pressure therapy, topical anti-bacterial ointments, systemic antibiotics, silver-based creams, and dressings. Furthermore, the methods of the present invention may be used in combination with physical therapy, occupational therapy, psychological therapy, diet, and exercise. In certain embodiments, the one or more additional treatment modalities comprises applying a topical medicament to the skin graft donor site prior to and/or following delivering said ultrasonic energy to said skin graft donor site.

In certain embodiments, the methods of the present invention are repeated so that ultrasonic energy is delivered to a donor site and/or a recipient site at least about twice per week for at least about two weeks. In other embodiments, ultrasonic energy is delivered to a donor site and/or a recipient site at least about twice per week for at least about 3, 4, or 5 weeks. In other embodiments, ultrasonic energy is delivered to the donor site and/or recipient site at least about three times per week for at least about 2, 3, 4, or 5 weeks. In other embodiments, ultrasonic energy is delivered to a donor site and/or recipient site once (e.g., the method comprises a single treatment).

In certain embodiments, the methods of the present invention comprise delivering ultrasonic energy to both the skin graft donor site and the skin graft recipient site. Such delivery includes providing ultrasonic energy to the donor site prior to and/or following skin graft removal and providing ultrasonic energy to the recipient site prior to and/or following skin graft transplantation. When both the donor site and the recipient site are treated, the invention contemplates that ultrasonic energy may be delivered to the two sites simultaneously or at differing times.

In certain embodiments, the therapeutic effect of the subject method is selected from one or more of decreasing bacteria at the donor site and/or the recipient site; promoting wound healing of a wound produced at the donor site; promoting wound healing of the recipient site; promoting epithelial cell proliferation at the donor site following graft removal; promoting epithelial cell proliferation at the recipient site following graft transplantation; promoting graft survival; decreasing inflammation at the donor site and/or recipient site; decreasing pain at the donor site and/or recipient site; decreasing or preventing scarring at the donor site and/or recipient site; decreasing or preventing biofilm formation; and decreasing the risk of infection.

In a third aspect, the present invention provides a method of preparing a skin graft donor site. The method comprises providing a transducer which can emit low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site prior to removing a skin graft from the donor site. The ultrasonic energy is delivered from a non-contact distance between a vibrating tip of the transducer and the skin graft donor site to provide a therapeutic effect.

Without being bound by theory, the emitted ultrasonic energy may provide a therapeutic effect via action at the tissue surface and/or by penetrating the treated patient tissue to provide a therapeutic effect beneath the tissue surface. In certain embodiments, the emitted ultrasonic energy provides a therapeutic effect at the tissue surface and/or beneath the tissue surface.

In certain embodiments, the ultrasonic energy is delivered to the skin graft donor site and to a periwound area adjacent to said skin graft donor site. In certain embodiments, the ultrasonic energy is delivered for a time proportional to the area of the skin graft donor site. In certain embodiments, the ultrasonic energy is delivered for a time proportional to the area of the skin graft donor site plus the periwound area.

In certain embodiments, the method further comprises harvesting a skin graft from said donor site and transplanting said skin graft to a recipient site. Exemplary skin grafts include, but are not limited to, full-thickness skin grafts and partial-thickness skin grafts. These methods may be used regardless of whether the skin graft is an autograft or an allograft.

In certain embodiments, the method further comprises delivering ultrasonic energy to the skin graft donor site following removing the skin graft from said site. The invention contemplates that ultrasonic energy may be delivered at multiple different time periods (e.g., the method is conducted numerous times as part of an overall treatment plan). Multiple treatments include any one or more of the following multiple different time periods: both prior to and following removing a skin graft from the donor site; multiple separate treatments prior to removing a skin graft from the donor site; multiple separate treatments following removing a skin graft from the donor site; multiple treatments prior to and/or following removing a skin graft from the donor site. However, in other embodiments, the method comprises a single treatment with ultrasonic energy prior to removal of the skin graft from the donor site.

In certain embodiments, ultrasonic energy is delivered to the skin graft donor site and the periwound area adjacent to the donor site.

In certain embodiments, the methods of the present invention further comprises delivering ultrasonic energy to the recipient site following transplantation of the skin graft to said recipient site.

In certain embodiments, the ultrasonic energy is delivered via a liquid spray. Delivery of ultrasonic energy via a liquid spray is sometimes referred to herein as “wet” delivery. When used “wet”, ultrasonic energy and the liquid spray are delivered to the treated tissue from a non-contact distance (e.g., without direct contact between the device used to deliver the ultrasonic energy and the treated patient tissue). By way of example, the liquid spray can be generated by delivering liquid to a distal portion of the transducer.

In certain embodiments, the ultrasonic energy is delivered in the absence of a liquid spray or coupling agent. Delivery of ultrasonic energy in the absence of a liquid spray or coupling agent is sometimes referred to herein as “dry” delivery.

In certain embodiments, the method is part of a therapeutic regimen combining one or more additional treatment modalities. Exemplary additional modalities include, but are not limited to, negative pressure therapy, topical anti-bacterial ointments, systemic antibiotics, silver-based creams, and dressings. Furthermore, the methods of the present invention may be used in combination with physical therapy, occupational therapy, psychological therapy, diet, and exercise. When used as part of a therapeutic regimen, the various treatment modalities can be administered/used a single time or multiple times and can be administered/used prior to, following, or during delivering of ultrasonic energy. In certain embodiments, the one or more additional treatment modalities comprises applying a topical medicament to the skin graft donor site prior to and/or following delivering said ultrasonic energy to said skin graft donor site.

In certain embodiments, the therapeutic effect of the subject method is selected from one or more of decreasing bacteria at the donor site; promoting wound healing of a wound produced at the donor site; promoting epithelial cell proliferation at the donor site following graft removal; decreasing or preventing scarring at the donor site; decreasing inflammation at the donor site; decreasing pain at the donor site; decreasing or preventing biofilm formation; and decreasing the risk of infection. Similarly, other exemplary therapeutic effects of performing the subject methods include one or more of decreasing bacteria at the donor site and/or the recipient site; promoting wound healing of a wound produced at the donor site; promoting wound healing of the recipient site; promoting epithelial cell proliferation at the donor site following graft removal; promoting epithelial cell proliferation at the recipient site following graft transplantation; promoting graft survival; decreasing inflammation at the donor site and/or recipient site; decreasing pain at the donor site and/or recipient site; decreasing or preventing scarring at the donor site and/or recipient site; decreasing or preventing biofilm formation; and decreasing the risk of infection.

In a fourth aspect, the invention provides a method for managing a skin graft donor site. The method comprises providing a transducer which can emit low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site following removing a skin graft from the donor site. The ultrasonic energy is delivered from a non-contact distance between a vibrating tip of the transducer and the skin graft donor site to provide a therapeutic effect.

Without being bound by theory, the emitted ultrasonic energy may provide a therapeutic effect via action at the tissue surface and/or by penetrating the treated patient tissue to provide a therapeutic effect beneath the tissue surface. In certain embodiments, the emitted ultrasonic energy provides a therapeutic effect at the tissue surface and/or beneath the tissue surface.

In certain embodiments, the ultrasonic energy is delivered to the skin graft donor site and to a periwound area adjacent to said skin graft donor site. In certain embodiments, the ultrasonic energy is delivered for a time proportional to the area of the skin graft donor site. In certain embodiments, the ultrasonic energy is delivered for a time proportional to the area of the skin graft donor site plus the periwound area. Exemplary skin grafts include, but are not limited to full-thickness skin grafts and partial-thickness skin grafts. Such skin grafts include autografts and allografts.

In certain embodiments, the ultrasonic energy is delivered via a liquid spray. Delivery of ultrasonic energy via a liquid spray is sometimes referred to herein as “wet” delivery. When used “wet”, ultrasonic energy and the liquid spray are delivered to the treated tissue from a non-contact distance (e.g., without direct contact between the device used to deliver the ultrasonic energy and the treated patient tissue). By way of example, the liquid spray can be generated by delivering liquid to a distal portion of the transducer, for example to a portion of the transducer tip.

In certain embodiments, the ultrasonic energy is delivered in the absence of a liquid spray or coupling agent. Delivery of ultrasonic energy in the absence of a liquid spray or coupling agent is sometimes referred to herein as “dry” delivery.

In certain embodiments, the method is part of a therapeutic regimen combining one or more additional treatment modalities. Exemplary additional modalities include, but are not limited to, negative pressure therapy, topical anti-bacterial ointments, systemic antibiotics, silver-based creams, and dressings. Furthermore, the methods of the present invention may be used in combination with physical therapy, occupational therapy, psychological therapy, diet, and exercise. When used as part of a therapeutic regimen, the various treatment modalities can be administered/used a single time or multiple times and can be administered/used prior to, following, or during delivering of ultrasonic energy. In certain embodiments, the one or more additional treatment modalities comprises applying a topical medicament to the skin graft donor site prior to and/or following delivering said ultrasonic energy to said skin graft donor site.

In certain embodiments, the method comprises multiple treatments of ultrasonic energy. For example, in certain embodiments the method comprises delivering ultrasonic energy at least about twice per week for at least two weeks. In other embodiments, the method comprises delivering ultrasonic energy at least three times per week for at least two weeks. In other embodiments, the method comprises a single treatment with ultrasonic energy.

In certain embodiments, the therapeutic effect of the subject method is selected from one or more of decreasing bacteria at the donor site; promoting wound healing of a wound produced at the donor site; promoting epithelial cell proliferation at the donor site following graft removal; decreasing or preventing scarring at the donor site; decreasing inflammation at the donor site; decreasing pain at the donor site; decreasing or preventing biofilm formation; and decreasing the risk of infection.

For any of the foregoing or following aspects and embodiments, the invention contemplates delivering low frequency ultrasonic energy. In certain embodiments, the ultrasonic energy is delivered at a frequency of approximately 20 kHz to 200 kHz. In other embodiments, the ultrasonic energy is delivered at a frequency of approximately 20 kHz to 100 kHz. In other embodiments, the ultrasonic energy is delivered at a frequency of approximately 20 kHz to 80 kHz or approximately 25 kHz to 60 kHz. In other embodiments, the ultrasonic energy is delivered at a frequency of approximately 30-50 kHz. In still other embodiments, the ultrasonic energy is delivered at a frequency of approximately 30-35 kHz, approximately 35-40 kHz, or approximately 40-45 kHz. In certain embodiments, the ultrasonic energy is delivered at a frequency of approximately 40 kHz.

For any of the foregoing or following aspects and embodiments, the invention contemplates delivering low frequency ultrasonic energy so as to provide a certain energy level to patient tissue. In certain embodiments, the ultrasonic energy level provided to patient tissue is approximately 0.1-1.0 watts/cm². In certain other embodiments, the ultrasonic energy level provided to patient tissue is approximately 0.1-0.7 watts/cm².

In certain embodiments, an effective amount of the delivered ultrasonic energy penetrates treated patient tissue to a depth of at least about 1 millimeter, at least about 2 millimeters, at least about 2.5 millimeters, at least about 2.75 millimeters, at least about 3 millimeters, or at least about 3.25 millimeters. In other embodiments, the delivered ultrasonic energy penetrates treated patient tissue to a depth of at least about 3.5 millimeters, at least about 3.75 millimeters, or at least about 4 millimeters. In certain embodiments, the ultrasonic energy penetrates treated patient tissue to a depth of more than about 4 millimeters (e.g., about 5, 6, 7, 8, 9, or even about 10 millimeters).

In certain embodiments, and regardless of whether and to what depth the emitted ultrasonic energy penetrates patient tissue, the emitted energy can provide a therapeutic effect at the tissue surface.

In certain embodiments, the delivered ultrasonic energy decreases the healing time of a wound at the recipient site and/or a wound at the donor site.

In certain embodiments, the delivered ultrasonic energy decreases pain, for example, pain associated with graft harvest from the donor site. Without being bound by theory, patients typically report that the pain associated with the donor site (e.g., with graft harvest and healing at the donor site) is greater than that experienced at the recipient site. Reduction in pain associated with the donor site can be evaluated relative to the pain experienced, on average, by patients whose treatment does not include treatment with low frequency, non-contact ultrasonic energy. Additionally or alternatively, reduction in pain may be evaluated based on the amount and frequency of pain medication requested or required to sufficiently manage patient pain relative, on average, to that needed by patient's whose treatment does not include low frequency, non-contact ultrasonic energy therapy. Such methods for evaluating reduction in pain are merely exemplary. Any standard method used by physicians and health care providers to evaluate pain and pain management can also be utilized.

In certain embodiments, the delivered ultrasonic energy decreases pain at one or both of the donor site and/or recipient site and is used as part of a regimen for reducing a patient's reliance on pain medication. A reduction in reliance on pain medication includes a reduction in the dosage of medication requested or required to control pain and/or a reduction in the frequency with which medication is requested or required to adequately control pain. A reduction in reliance on pain medication may also include a shift from narcotic-based pain medications to non-narcotic or other over the counter pain medication (for example, a shift from morphine to ibuprofen).

In certain embodiments, ultrasonic treatment reduces scarring at one or both of the donor and/or recipient site. Reduction in scarring can be assessed by comparison to the level of scarring typically observed following other similar procedures performed in the absence of ultrasonic treatment. Reduction in scarring can be assessed based on previous procedures performed on the same person or procedures performed on different patients.

In certain embodiments, the delivered ultrasonic energy decreases the rate of skin graft failure. In certain embodiments of any of the foregoing, the delivered ultrasonic energy promotes skin graft survival and/or decreases the healing time of the recipient site.

In certain embodiments, the low frequency ultrasonic energy delivered from a non-contact distance is non-thermal. In other words, delivery of the ultrasonic energy (and optionally liquid spray) does not cause a significant increase in the temperature of the treated patient tissue (e.g., does not increase the temperature of the treated patient tissue by more than approximately 1° F.).

The invention contemplates all suitable combinations of one or more of any of the foregoing or following aspects or embodiments of the claimed invention. Further, exemplary systems and ultrasound treatment devices suitable for delivering ultrasonic energy to a skin graft donor site and/or a skin graft recipient site are described in detail in U.S. Pat. No. 6,569,099, and U.S. patent application Ser. Nos. 11/473,934, 10/409,272, 10/815,384, and 12/006,739. The disclosures of each of these patents and patent applications are hereby incorporated by reference in their entirety.

### BRIEF DESCRIPTION OF THE DRAWINGS

FIG. 1 shows an exemplary system for delivering ultrasonic energy to a patient.

FIG. 2 shows an exemplary ultrasound transducer for delivering ultrasonic energy to a patient. The figure depicts an exemplary transducer, an applicator nozzle, and a fluid source.

FIG. 3 shows another exemplary system for delivering ultrasonic energy to a patient. The figure depicts a system, which includes drive electronics and software for operating the device and providing information to the operator via a graphical user interface; an ultrasonic transducer; an applicator nozzle; and a fluid source.

FIG. 4 shows another exemplary system for delivering ultrasonic energy to a patient. The figure depicts a system, which includes drive electronics and software for operating the device, controlling fluid flow, and providing information to the operator via a graphical user interface; an ultrasonic transducer; an applicator nozzle; and a fluid source.

### DETAILED DESCRIPTION OF THE INVENTION

### (i) Overview

The present invention provides methods for managing skin grafting at a donor site and/or recipient site. The present invention provides improved methods for performing skin grafting by delivering low frequency ultrasonic energy from a non-contact distance to a donor site and/or a recipient site. Delivering low frequency ultrasonic energy prior to, during and/or subsequent to graft harvest (when referring to the donor site) and/or graft placement (when referring to the recipient site) has numerous benefits. By way of example, providing an effective amount of ultrasonic energy (in the presence or absence of a liquid mist) has therapeutic effects including one or more of the following: kill bacteria, decrease bacterial burden, decrease the presence of a biofilm, reduce pain, decrease inflammation, promote wound healing, promote epithelial cell proliferation and/or differentiation, prevent or reduce contraction, and prevent or reduce scarring.

In a first aspect, the invention provides a method of reducing pain associated with removal of a skin graft from a skin graft donor site. The method comprises providing a transducer which can emit low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site following removal of a skin graft from said site. The ultrasonic energy is delivered from a non-contact distance between a vibrating tip of the transducer and the skin graft donor site. The delivered ultrasonic energy provides a therapeutic effect to reduce pain associated with removal of a skin graft from said skin graft donor site. Without being bound by theory, the emitted ultrasonic energy may provide a therapeutic effect via action at the tissue surface and/or by penetrating the treated patient tissue to provide a therapeutic effect beneath the tissue surface. In certain embodiments, the emitted ultrasonic energy provides a therapeutic effect at the tissue surface and/or beneath the tissue surface.

In a second aspect, the invention provides a method of preparing tissue for skin grafting. The method comprises providing a transducer which emits low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site and/or skin graft recipient site, and the ultrasonic energy is delivered from a non-contact distance between a vibrating tip of the transducer and treated patient tissue. The delivered ultrasonic energy acts at the tissue surface and/or penetrates the treated patient tissue (e.g., penetrates tissue of the skin graft donor site and/or skin graft recipient site) to provide a therapeutic effect.

In a third aspect, the present invention provides a method of preparing a skin graft donor site. The method comprises providing a transducer which emits low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site prior to removing a skin graft from the donor site. The ultrasonic energy is delivered from a non-contact distance between a vibrating tip of the transducer and the skin graft donor site. The delivered ultrasonic energy acts at the tissue surface and/or penetrates the treated patient tissue to provide a therapeutic effect.

In a fourth aspect, the invention provides a method for managing a skin graft donor site. The method comprises providing a transducer which emits low frequency ultrasonic energy. An example of a suitable transducer is provided by an ultrasound therapy device that comprises a transducer, wherein the transducer can emit low frequency ultrasonic energy. The emitted ultrasonic energy is delivered to a skin graft donor site following removing a skin graft from the donor site. The ultrasonic energy is delivered from a non-contact distance between a vibrating tip of the transducer and the skin graft donor site. The delivered ultrasonic energy acts at the tissue surface and/or penetrates the treated patient tissue to provide a therapeutic effect.

For any of the foregoing aspects of the invention, any of the foregoing or following embodiments are contemplated.

In certain embodiments, the method comprises delivering ultrasonic energy to a skin graft donor site. In certain embodiments, the ultrasonic energy is delivered prior to removing a skin graft from the donor site. In other embodiments, the ultrasonic energy is delivered following removing a skin graft from the donor site. In still other embodiments, the ultrasonic energy is delivered over any one or more of the following multiple different time periods: both prior to and following removing a skin graft from the donor site; multiple separate treatments prior to removing a skin graft from the donor site; multiple separate treatments following removing a skin graft from the donor site; multiple treatments prior to and/or following removing a skin graft from the donor site. Similarly, a single treatment at the donor site prior to and/or following skin graft removal is contemplated. Optionally, ultrasonic energy may also be delivered to the recipient site prior to and/or following graft transplantation. Multiple treatments at the recipient site, as well as single treatments are contemplated. In certain embodiments, following graft transplantation to the recipient site, treatment with ultrasonic energy is delayed (or suspended if used prior to transplantation) for approximately five, six, seven, eight, nine, ten days, or even about two weeks to allow the graft to “take” prior to beginning or resuming ultrasonic energy treatment.

In certain embodiments, the ultrasonic energy is delivered via a liquid spray. Delivery of ultrasonic energy via a liquid spray is sometimes referred to herein as “wet” delivery. When used “wet”, ultrasonic energy and the liquid spray are delivered to the treated tissue from a non-contact distance (e.g., without direct contact between the device used to deliver the ultrasonic energy and the treated patient tissue). By way of example, the liquid spray can be generated by delivering liquid to a distal portion of the transducer, for example to a portion of the transducer tip.

In certain embodiments, the ultrasonic energy is delivered in the absence of a liquid spray or coupling agent. Delivery of ultrasonic energy in the absence of a liquid spray or coupling agent is sometimes referred to herein as “dry” delivery. As with “wet” delivery, the ultrasonic energy is delivered from a non-contact distance.

In certain embodiments, the method further comprises harvesting a skin graft from the donor site and transplanting the skin graft to a recipient site. Exemplary skin grafts include, but are not limited to, full-thickness skin grafts and partial-thickness skin graft. These methods may be used regardless of whether the skin graft is an autograft or an allograft. Accordingly, the invention contemplates embodiments in which the donor site and the recipient site are on the same patient, as well as embodiments in which the donor site and recipient site are on different patients. In certain embodiments, although the graft is an allograft, the donor is the recipient's identical twin.

In certain embodiments, the method is part of a therapeutic regimen combining one or more additional treatment modalities. Exemplary additional modalities include, but are not limited to, negative pressure therapy, topical anti-bacterial ointments, systemic antibiotics, silver-based creams, and dressings. Furthermore, the methods of the present invention may be used in combination with physical therapy, occupational therapy, psychological therapy, diet, and exercise. When used as part of a therapeutic regimen, the various treatment modalities can be administered/used a single time or multiple times and can be administered/used prior to, following, or during delivering of ultrasonic energy. In certain embodiments, the graft is an allograft, and an additional treatment modality includes administration of an immunosuppressive agent or other drug to help prevent graft rejection.

In certain embodiments, the methods of the present invention are repeated so that ultrasonic energy is delivered to the donor site and/or recipient site at least about twice per week for at least about two weeks. In other embodiments, ultrasonic energy is delivered to the donor site and/or recipient site at least about twice per week for at least about 3, 4, or 5 weeks. In other embodiments, ultrasonic energy is delivered to the donor site and/or recipient site at least about three times per week for at least about 2, 3, 4, or 5 weeks. In still other embodiments, the method comprises a single treatment. Such single treatments include a single treatment of the donor site prior to and/or following removal of the skin graft.

In certain embodiments, the methods of the present invention comprise delivering ultrasonic energy to both the skin graft donor site and the skin graft recipient site. Such delivery includes providing ultrasonic energy to the donor site prior to and/or following skin graft removal and providing ultrasonic energy to the recipient site prior to and/or following skin graft transplantation. When both the donor site and the recipient site are treated, the invention contemplates that ultrasonic energy may be delivered to the two sites simultaneously or at differing times.

In certain embodiments, the therapeutic effect of the subject method is selected from one or more of decreasing bacteria at the donor site and/or the recipient site; promoting wound healing of a wound produced at the donor site; promoting wound healing of the recipient site; promoting epithelial cell proliferation at the donor site following graft removal; promoting epithelial cell proliferation at the recipient site following graft transplantation; promoting graft survival; decreasing inflammation at the donor site and/or recipient site; decreasing pain at the donor site and/or recipient site; decreasing or preventing scarring at the donor site and/or recipient site; reduce contraction; decreasing or preventing biofilm formation; and decreasing the risk of infection.

In certain embodiments, the ultrasonic energy is delivered to the skin graft donor site and to a periwound area adjacent to said skin graft donor site. In certain embodiments, the ultrasonic energy is delivered for a time proportional to the area of the skin graft donor site. In certain embodiments, the ultrasonic energy is delivered for a time proportional to the area of the skin graft donor site plus the periwound area.

In certain embodiments, the method further comprises harvesting a skin graft from said donor site and transplanting said skin graft to a recipient site. Exemplary skin grafts include, but are not limited to, full-thickness skin grafts and partial-thickness skin grafts. These methods may be used regardless of whether the skin graft is an autograft or an allograft.

In certain embodiments, the method further comprises delivering ultrasonic energy to the skin graft donor site following removing the skin graft from said site. The invention contemplates that ultrasonic energy may be delivered at multiple different time periods (e.g., the method is conducted numerous times as part of an overall treatment plan). Multiple treatments include any one or more of the following multiple different time periods: both prior to and following removing a skin graft from the donor site; multiple separate treatments prior to removing a skin graft from the donor site; multiple separate treatments following removing a skin graft from the donor site; multiple treatments prior to and/or following removing a skin graft from the donor site.

In certain embodiments, ultrasonic energy is delivered to the skin graft donor site and the periwound area adjacent to the donor site.

In certain embodiments, the methods of the present invention further comprise delivering ultrasonic energy to the recipient site following transplantation of the skin graft to said recipient site.

In certain embodiments, the therapeutic effect of the subject method is selected from one or more of decreasing bacteria at the donor site; promoting wound healing of a wound produced at the donor site; promoting epithelial cell proliferation at the donor site following graft removal; decreasing or preventing scarring at the donor site; decreasing inflammation at the donor site; decreasing pain at the donor site; decreasing or preventing biofilm formation; and decreasing the risk of infection. Similarly, other exemplary therapeutic effects of performing the subject methods include one or more of decreasing bacteria at the donor site and/or the recipient site; promoting wound healing of a wound produced at the donor site; promoting wound healing of the recipient site; promoting epithelial cell proliferation at the donor site following graft removal; promoting epithelial cell proliferation at the recipient site following graft transplantation; promoting graft survival; decreasing inflammation at the donor site and/or recipient site; decreasing pain at the donor site and/or recipient site; decreasing or preventing scarring at the donor site and/or recipient site; decreasing or preventing biofilm formation; and decreasing the risk of infection.

In certain embodiments, the ultrasonic energy is delivered to the skin graft donor site and to a periwound area adjacent to said skin graft donor site. In certain embodiments, the ultrasonic energy is delivered for a time proportional to the area of the skin graft donor site. In certain embodiments, the ultrasonic energy is delivered for a time proportional to the area of the skin graft donor site plus the periwound area. Exemplary skin grafts include, but are not limited to full-thickness skin grafts and partial-thickness skin grafts. Such skin grafts include autografts and allografts.

In certain embodiments, the method comprises multiple treatments of ultrasonic energy. For example, in certain embodiments the method comprises delivering ultrasonic energy at least about twice per week for at least two weeks.

For any of the foregoing or following aspects and embodiments, the invention contemplates delivering low frequency ultrasonic energy. In certain embodiments, the ultrasonic energy is delivered at a frequency of approximately 20 kHz to 200 kHz. In other embodiments, the ultrasonic energy is delivered at a frequency of approximately 20 kHz to 100 kHz. In other embodiments, the ultrasonic energy is delivered at a frequency of approximately 20 kHz to 80 kHz or approximately 25 kHz to 60 kHz. In other embodiments, the ultrasonic energy is delivered at a frequency of approximately 30-50 kHz. In still other embodiments, the ultrasonic energy is delivered at a frequency of approximately 30-35 kHz, approximately 35-40 kHz, or approximately 40-45 kHz. In certain embodiments, the ultrasonic energy is delivered at a frequency of approximately 40 kHz.

For any of the foregoing or following aspects and embodiments, the invention contemplates delivering low frequency ultrasonic energy so as to provide a certain energy level to patient tissue. In certain embodiments, the ultrasonic energy level provided to patient tissue is approximately 0.1-1.0 watts/cm². In certain other embodiments, the ultrasonic energy level provided to patient tissue is approximately 0.1-0.7 watts/cm².

In certain embodiments, an effective amount of the delivered ultrasonic energy penetrates treated patient tissue to a depth of at least about 1 millimeter, at least about 2 millimeters, at least about 2.5 millimeters, at least about 2.75 millimeters, at least about 3 millimeters, or at least about 3.25 millimeters. In other embodiments, the delivered ultrasonic energy penetrates treated patient tissue to a depth of at least about 3.5 millimeters, at least about 3.75 millimeters, or at least about 4 millimeters. In certain embodiments, the ultrasonic energy penetrates treated patient tissue to a depth of more than about 4 millimeters (e.g., more than about 4, 5, 6, 7, 8, 9, or even about 10 millimeters). Without being bound by theory, one of skill in the art will appreciate that when all or a portion of the therapeutic benefit of ultrasonic energy is due to penetration of the energy below the tissue surface, that this indicates that an effective amount of energy penetrates to an effective depth. However, such an effective amount need not be and is likely not the same energy level as that which initially contacts the tissue surface.

In certain embodiments, the delivered ultrasonic energy decreases the healing time of one or both of a wound at the recipient site or a wound at the donor site.

In certain embodiments, the delivered ultrasonic energy decreases pain, for example, pain associated with graft harvest from the donor site.

In certain embodiments, the delivered ultrasonic energy decreases the rate of skin graft failure. In certain embodiments of any of the foregoing, the delivered ultrasonic energy promotes skin graft survival and/or decreases the healing time of the recipient site.

In certain embodiments, the ultrasound energy delivered is low frequency ultrasound energy. In certain embodiments, the ultrasound energy delivered is low intensity.

In certain embodiments, low frequency ultrasound is delivered (in the presence or absence of a liquid spray) from a non-contact distance and without causing a substantial increase in the temperature of the treated tissue. In certain embodiments, low frequency ultrasound is delivered in the presence of a liquid spray from a non-contact distance and without causing a substantial increase in the temperature of the treated tissue or in the temperature of the liquid spray.

In certain embodiments of any of the foregoing, ultrasound energy is delivered from a non-contact distance “dry” (e.g., in the absence of a liquid coupling medium or liquid mist). In certain embodiments of any of the foregoing, ultrasound energy is delivered from a non-contact distance in the presence of a liquid mist (“wet”). The liquid mist is generated by contacting, dripping, or otherwise delivering a liquid to a portion of a vibrating ultrasound transducer, for example, a portion of the transducer tip, to create a spray. The spray and the ultrasound energy are directed to the patient tissue. When ultrasound energy is delivered “wet”, the liquid may be an inert or substantially inert liquid such as saline solution, oil, Ringer's solution, sterile water, and the like. The liquid may also be or contain a therapeutic medicament including, but not limited to, a growth factor, antibiotic, antifungal, antimicrobial, analgesic, anti-inflammatory, hypochlorous acid, or angiogenesis promoting agent. In certain embodiments, the fluid spray produced has a substantially uniform particle size. Exemplary fluids include, but are not limited to, sterile water, oxygenated water, saline solution, oil, or other isotonic or hypertonic solution. In certain embodiments, the fluid does not contain a therapeutic drug (e.g., the fluid is substantially free from a drug). In certain other embodiments, the fluid further includes one or more therapeutic drugs such as antibiotics, anti-fungals, anti-virals, growth factors, analgesics, narcotics, and the like. When the fluid includes a therapeutic drug, the drug may be formulated in any of the foregoing fluids (e.g., water, saline, etc), or the drug may be formulated in another pharmaceutically acceptable carrier appropriate for the formulation of the particular drug. In certain embodiments, the fluid (whether including a therapeutic drug or free from therapeutic drug) further includes one or more preservatives appropriate for extending the shelf-life of the fluid. In one embodiment of any of the foregoing, the fluid (whether including a therapeutic drug or free from therapeutic drug) is sterile (e.g., the fluid is sterilized prior to or after it is added to the bottle or other fluid container).

In certain embodiments, ultrasound energy is delivered to the recipient and/or donor site prior to graft harvest and/or graft placement. In certain embodiments, ultrasound energy is delivered to the recipient and/or donor site during graft harvest and/or graft placement. In certain embodiments, ultrasound energy is delivered to the recipient and/or donor site subsequent to graft harvest and/or graft placement. In certain embodiments, ultrasound energy is delivered multiple time points (e.g., before and after graft harvest and/or graft placement; numerous separate treatments before and/or after graft harvest and/or graft placement).

In certain embodiments, both the donor site and periwound space are treated with ultrasound energy. In certain embodiments, both the recipient site and periwound space are treated with ultrasound energy.

In certain embodiments, the method comprises delivering ultrasound energy at a non-contact distance.

In certain embodiments, the method for treating a patient comprises multiple treatments. For example, patients may receive doses of ultrasound two or more times per week, for one, two, three, four, or more than four weeks. The appropriate number of treatments, and the duration of each treatment, can be determined by a health care provider based on, for example, the particular condition being treated, the severity of the condition, and the overall health of the patient. Furthermore, the health care provider can determine whether treatment should be “wet” or “dry”.

In certain embodiments, the low frequency ultrasound energy increases the viability of the skin graft and reduces or prevents skin graft failure at the recipient site.

The invention contemplates all suitable combinations of one or more of any of the foregoing or following aspects or embodiments of the claimed invention. Further, exemplary systems and ultrasound treatment devices suitable for delivering ultrasonic energy to one or more of a skin graft donor site or a skin graft recipient site are described in detail in U.S. Pat. No. 6,569,099, and U.S. patent application Ser. Nos. 11/473,934, 10/409,272, 10/815,384, and 12/006,739. The disclosures of each of these patents and patent applications are hereby incorporated by reference in their entirety.

### (ii) Definitions

Unless defined otherwise, all technical and scientific terms have the same meaning as is commonly understood by one of skill in the art to which this invention belongs.

The articles “a” and “an” are used herein to refer to one or to more than one (i.e., to at least one) of the grammatical object of the article. By way of example, “an element” means one element or more than one element.

The term “donor site” refers to the tissue and region of the body that is the source of tissue for a skin graft. As used herein, and unless otherwise specified, the term donor site is used to refer to the source area of the skin graft both prior to and following skin graft removal. By way of example, if a skin graft is going to be harvested from an area of a patient's upper arm, that region is referred to as the donor site. Prior to removal of the graft, the term donor site refers to the area from which the graft will be harvested and includes the intact skin and tissue existing at that site. Following graft removal, the term donor site refers to the area from which the graft was harvested, as well as the wound generated at that site following graft removal.

The term “recipient site” refers to the region of the body to which a skin graft is transplanted. As used herein, and unless otherwise specified, the term recipient site is used to refer to this region both prior to and following skin-graft transplantation.

By “treatment” is meant to refer to a session during which ultrasonic energy is delivered to patient tissue. Typically, a treatment is at least 1 minute in length.

The term “low frequency”, with respect to ultrasound energy, refers to frequencies less than approximately 500 kHz.

The term “non-contact” is used to refer to methods for delivering ultrasonic energy to patient tissue without direct contact between the treated patient tissue (e.g., the treated donor site or the treated recipient site, as applicable). When non-contact methods for delivering ultrasonic energy are used, the ultrasound transducer (including the transducer tip portion) does not contact (directly or via a coupling gel) the treated patient tissue. The non-contact distance can be measured as the distance between the distal most surface of the ultrasound transducer tip and the treated patient tissue or the non-contact distance can be measured as the distance between the distal most surface of an applicator nozzle and treated patient tissue. Exemplary non-contact distances are at least about 0.1 inches (2.5 mm) or from about 2.5 mm to about 51 cm or from about 15 mm to about 25 mm. However, recitation of an approximate non-contact distance does not indicate that the exact distance is maintained for the entire treatment time. More importantly, the term non-contact is used to indicate that there is no contact with the treated tissue. However, it is possible and permissible that components of the applicator or device may contact patient tissue that is not the intended target of treatment. For example, to facilitate delivery of the ultrasound energy, a handle of the device may be affixed to a patient's arm, thereby alleviating the need for an operator to hold the device throughout treatment. Such contact with other patient tissue that is not the intended target of treatment does not alter the characterization of the treatment as “non-contact”.

The term “applicator” and “applicator nozzle” are used interchangeably to refer to an optional portion of an ultrasound therapy device. When present, the nozzle shields the transducer tip and prevents inadvertent contact with the transducer tip when the device is in operation. Additionally, the applicator nozzle can be used as part of the mechanism for delivering fluid to a portion of the transducer and/or as part of the mechanism for directing the delivered ultrasonic energy and/or liquid spray to patient tissue. Exemplary applicator nozzles are depicted herein. However, other applicator nozzles, as well as transducer assembly designs that do not include an applicator nozzle can similarly be used.

The terms “ultrasonic energy” and “ultrasound energy” are used interchangeably herein.

### (iii) Systems, Devices and Methods for Delivering Ultrasonic Energy

Numerous systems and devices for delivering ultrasonic energy are available. Such existing devices, as well as modifications and improvements thereof, are exemplary of systems and devices that may be used to deliver low frequency ultrasonic energy to patient tissue. In certain embodiments, low frequency ultrasonic energy is delivered from a non-contact distance and without direct contact with treated patient tissue. For example, the low frequency ultrasonic energy (in the presence or absence of liquid spray) is delivered from a non-contact distance between the treated patient tissue and the transducer tip of the ultrasound device and/or the applicator nozzle.

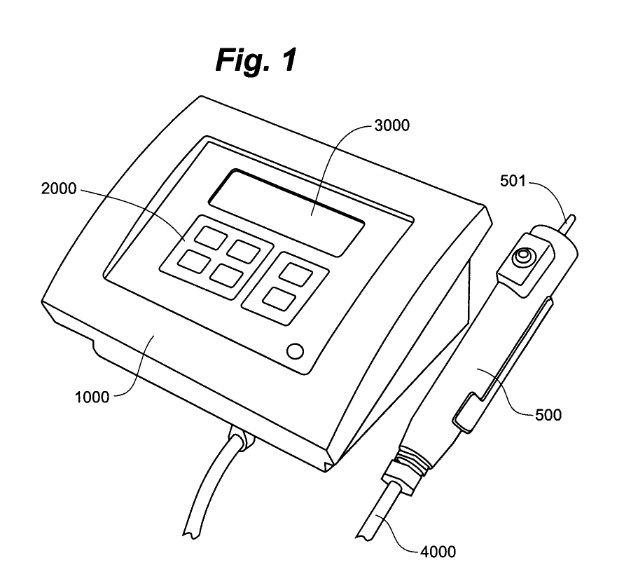

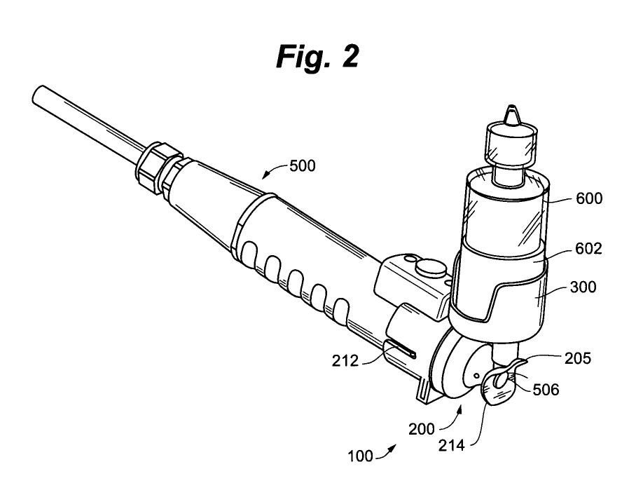

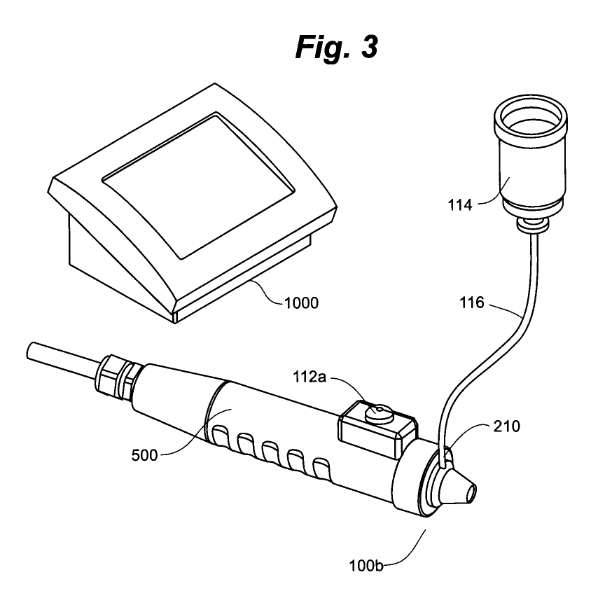

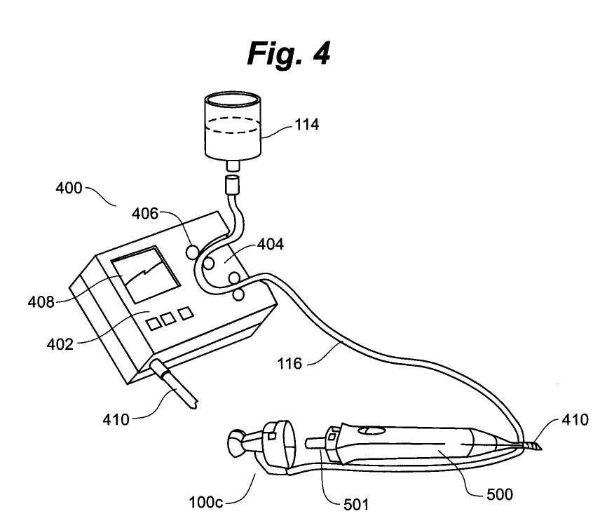

FIG. 1 depicts an exemplary system for delivering ultrasonic energy. An exemplary ultrasound therapy device includes a transducer assembly 500 operatively connected via a connector 4000 to a generator 1000. As described herein, the ultrasound therapy device may further include an applicator that can be interconnected to the transducer assembly 500, thereby shielding the transducer tip portion 501. Exemplary applicator designs are set forth in FIG. 2 (applicator 100), FIG. 3 (applicator 100b), and FIG. 4 (applicator 100c). However, particularly when ultrasonic energy is delivered “dry”, the presence of an applicator is optional.

Briefly, the generator 1000 includes the components necessary to supply power to the transducer assembly 500, and also contains a control panel 2000, and a graphical user interface (GUI) 3000 for displaying information helpful to the operator. The generator 1000 consists of three major functional sections: the AC MAINS, the main board, and the GUI board. The local AC MAINS is connected to an appliance inlet with a hospital grade detachable power cord. The appliance inlet is a power entry module listed for medical applications. In certain embodiments, the appliance inlet is a power entry module with an 115V/230V voltage selection, and is designed to operate on 115Vac and 60 Hz (e.g., for operation in North America) or 230 Vac and 50 Hz (e.g., for operation in Europe).

The MAIN board converts the secondary output voltage from the MAINS transformer to the low voltage power rails for the internal electronics and the drive voltage for the drive electronics to the transducer assembly. The MAIN board contains a microprocessor that controls, measures, and monitors the drive electronics. The transducer assembly connects to the MAIN board. The microprocessor, referred to as the engine, monitors the performance of the system and communicates the information to a second microprocessor located on the GUI board. In certain embodiments, the engine communicates to the second microprocessor via a RS-232 communication link. In certain embodiments, the electronics drive the ultrasound portion of the drive electronics with a push-pull converter that has a feedback loop with a Phase Locked Loop (PLL) to track the center frequency of the ultrasound components.

The GUI board provides the graphical user interface for the operator. A custom membrane switch panel with, for example 6 keys, allows the operator to select the functions and operating parameters of the system. A purchased graphical LCD display, connected to the GUI board, can be used to display information to the operator. For example, information about the system's status, mode of operation, and treatment time can be displayed via the GUI. The GUI may have a back light generator for the LCD on it. The GUI microprocessor runs the system by controlling the human interface and running various algorithms to control the operation of the system. For example, a treatment algorithm can be run on the GUI microprocessor. In certain embodiments, the system may include one or more of a timer to record total treatment time, a timer to count-down from a selected treatment time to zero, and an alarm to indicate that the total treatment time has elapsed or that there is a problem with some component of the device.

FIG. 1 also depicts an example of a transducer assembly 500. As depicted, only the transducer tip portion 501 is visible. The remainder of the transducer is contained within the plastic casing of the assembly. As depicted, the transducer tip portion 501 is uncovered. In operation, the transducer tip portion 501 may be shielded with, for example, an applicator nozzle. Exemplary applicator nozzles 100, 100b, and 100c are depicted in FIGS. 2-4. When used, an applicator nozzle helps prevent inadvertent contact of either the operator or the patient with the vibrating tip portion of the transducer. Additionally, an applicator nozzle can be used as part of the mechanism for directing the delivered ultrasonic energy to patient tissue. When the system is used “wet”, the applicator nozzle can also be used to deliver fluid to the transducer tip portion and to direct the delivered ultrasonic energy and the fluid spray to patient tissue.

The system depicted in FIG. 1 is currently sold by Celleration, Inc. as part of the MIST Therapy® System.

FIG. 2 shows an example of a portion of a system for delivering ultrasonic energy. Specifically, FIG. 2 shows a transducer assembly 500 interconnected to one embodiment of an applicator nozzle 100. The transducer assembly can be operatively interconnected to a generator, for example generator 1000 shown in FIG. 1.

As depicted, the transducer assembly 500 and applicator nozzle 100 are ready to be used “wet”. Specifically, a fluid bottle 600 containing fluid 602 is interfitted to a portion of the applicator nozzle so that fluid can be delivered to the transducer tip portion, and so that ultrasonic energy and a fluid spray can be delivered to patient tissue. As depicted, a fluid bottle 600 is interfitted to a cup portion 300 of the applicator nozzle. As shown, fluid delivery to the transducer would largely be gravity driven. However, pressure delivery methods, peristaltic pumps, fluid cartridges affixed directly to or housed within the transducer assembly, and the like are similarly contemplated. An alternative mechanism for providing fluid to the transducer is via a sock, membrane, film, or other means to wick fluid from a fluid container or fluid line to all or a portion of the transducer. In certain embodiment's, fluid is delivered to one or more regions along the transducer tip portion.

In certain embodiments, an applicator, as described herein, is interconnected with an ultrasound therapy device and used to deliver ultrasound energy (in the presence or absence of a liquid spray) to patient tissue. When used in this manner, the ultrasound energy (and liquid spray, if present) is delivered without contact between the applicator and the patient tissue being treated. In other words, the ultrasound energy (and liquid spray, if present) are delivered from a non-contact distance. Once delivered, the ultrasound energy acts at the cell surface and/or penetrates the treated tissue to provide a therapeutic effect.

The transducer assembly 500 and applicator nozzle 100 depicted in FIG. 2 is currently sold by Celleration, Inc. as part of the MIST Therapy® System. As depicted, applicator 100 generally includes a nozzle 200 and a cup 300. However, applicator designs that exclude the cup 300 can be readily used.

When included in the applicator design, the cup 300 may be designed to hold at least a portion of a bottle 600 therein. The bottle 600 generally holds a fluid 602, which may be saline. The fluid may alternatively be sterile water or some other isotonic or hypertonic solution or combination of solutions. The fluid may consist entirely or essentially of the saline or other similar solution, or the fluid may optionally include a therapeutic drug. The fluid may optionally be sterilized.

The applicator 100 is mechanically connectable with a transducer assembly 500 of an ultrasound therapy device. When activated, the transducer assembly 500 produces ultrasonic waves having a frequency and capable of delivering ultrasonic energy to patient tissue.

The proximal portion of the nozzle 200 slides over a distal portion of the transducer assembly 500. The plurality of aligning slots 212 (illustrated as two slots) of the nozzle 200 engage with a plurality of aligning pins of the transducer assembly 500. When connected, the distal end 506 of a transducer tip portion of the transducer assembly 500 may extend distally of the distal opening 214 of the nozzle 200 but not to a location that is distal of the tip 205 of the nozzle 200.

The fluid 602 to be sprayed and provided within the bottle 600 can be any appropriate carrier, such as saline, water (regular or distilled), or oil to be applied to tissue, such as a vegetable, peanut, or canola oil, optionally with a soluble pharmaceutical (e.g., an antibiotic), antiseptic, conditioner, surfactant, emollient, or other active ingredient. The fluid 602 can also be a combination of two or more fluids and/or substances having microscopic particles, such as powder and the like. Exemplary fluids include, but are not limited to, sterile water, saline solution, oil, oxygenated water, or other isotonic or hypertonic solutions. Exemplary fluids may, in certain embodiments, further include drugs (e.g., therapeutic agents) such as antibiotics, anti-fungals, anti-virals, growth factors, analgesics, narcotics, and the like, formulated in any of the foregoing fluids or in other pharmaceutically acceptable fluids appropriate for the formulation of the particular drug. However, in certain embodiments, the fluid does not include a drug. The fluid may be sterilized so that, in use, a spray of a sterile solution can be administered to patients.

It is envisioned for the bottle 600 of the present disclosure to be eliminated and/or replaced with another structure for delivering the fluid 602 to the transducer assembly 500, such as a fluid bag (not shown). In such an embodiment, the fluid 602 may optionally be delivered to the transducer assembly 500 in a pressurized state. Desirably, the pressurized fluid 602 in such an embodiment may be approximately equal to the pressure of the fluid 602 exiting the bottle 600, as in the previous embodiment.

FIG. 3 shows another example of a portion of a system for delivering ultrasonic energy. Specifically, FIG. 3 shows a generator 1000, a transducer assembly 500, and an alternative design for an applicator nozzle 100b.

FIG. 3 depicts an applicator 100b. As depicted the applicator 100b is interconnected to a transducer assembly 500. The applicator 300 is also interconnected to a fluid source via a flexible tubing.

FIG. 3 also show a switch 112a that may control one or more of the power supplied to the transducer assembly 500, the flow of fluid, or the fluid flow rate. Also shown is a fluid source 114 and tubing 116 that interconnects the fluid source 114 to the applicator 300 via a connector 210. As depicted, the connector comprises an opening in communication with the interior of the applicator 100b, thereby providing a conduit to deliver fluid to a portion of the transducer.

FIG. 4 shows another example of a portion of a system for delivering ultrasonic energy. Specifically, FIG. 4 shows a pump-generator 400, a transducer assembly 500, and an alternative design for an applicator nozzle 100c.

As depicted the transducer assembly and applicator are interconnected to a fluid source 114 via flexible tubing 116. The applicator 100c is depicted just prior to interconnection to the transducer assembly 500. The transducer tip portion 501 is visible. Once the applicator 100c is interfitted to the transducer assembly 500, the transducer tip portion 501 will be shielded, thereby preventing inadvertent contact with the transducer tip portion 501.

In this depiction, the pump-generator 400 includes additional mechanisms for controlling fluid delivery to the transducer assembly 500, the transducer tip portion 501, and the applicator 100. The depicted system provides an example of a fluid delivery mechanism that is not gravity fed, but rather under direct control of the user. The use of a peristaltic pump, such as the pump depicted, permits additional control over the rate at which fluid is delivered to the transducer.

An exemplary peristaltic pump at least includes a rotor and rollers or other tube-engaging members movable within a housing relative to the clamped flexible tubing. A peristaltic pump typically includes between four to six rollers. The rollers compress the clamped flexible tubing. As the rotor turns, the part of the tube under compression gets pinched and the pinching motion forces the fluid to move through the tube. The rollers relax the clamped flexible tubing as the rotor turns and the flexible tubing opens to its original state to induce fluid flow. FIG. 4 shows a fluid container 114, a tubing 116, an applicator 100c, and a generator-pump unit 400. The generator-pump unit 400 includes, among other things, a generator portion 402, a pump portion 404, multiple rollers 406, an LCD display 408, and a connection inlet 410. The generator portion 402 may automate the fluid to enter the nozzle by, for example, regulating a valve (not shown) coupled to the tubing 116. In addition, the pressure applied to the fluid may be automatically maintained by the generator 402 based on values supplied by the user from a user interface, such as a dial, coupled to the generator 402. In addition, the generator 402 may report to the user the monitored pressure readings in the LCD display 404 of the generator 402. Although not shown, the generator-pump unit 400 may include an outer cover to protect the rollers 406 and the flexible tubing. In certain embodiments, the generator-pump unit 400 is fully integrated such that it performs all of the functions of the generator 1000 depicted in FIG. 1.

Although not depicted in the foregoing figures, when used “wet”, fluid flows into the nozzle and is delivered to a vibrating transducer tip portion 501. Fluid delivery can be, for example, gravity driven or mechanically or otherwise controlled. The fluid source can be separate from or integrated within the generator and/or transducer assembly. Fluid delivery can be along all or a portion of the transducer tip portion, including to a distal portion of the tip portion. Fluid is dripped, flowed, wicked, or otherwise applied to all or a portion of the transducer tip portion, including to a plurality of sections of the transducer tip portion. Ultimately, in operation, fluid contacts all or a portion of the vibrating transducer tip portion, thus generating a spray. The fluid spray and ultrasonic energy are delivered to patient tissue. Fluid contacts the transducer tip portion and ultrasonic energy and a fluid spray are delivered from the distal end of the applicator nozzle, when such nozzle is present, or from the distal end of the transducer.

FIGS. 1-4 are merely exemplary of systems and devices that can be used to deliver ultrasonic energy. Delivery of low frequency ultrasound energy using other systems and devices is similarly contemplated.

Regardless of whether the foregoing or functionally related or differing devices are used, and regardless of whether used “wet” or “dry”, ultrasonic energy is delivered to patient tissue without direct contact between the transducer tip and/or applicator nozzle and the treated patient tissue. In certain embodiments, ultrasonic energy is delivered “dry” and the system does not include an applicator nozzle. In certain embodiments, ultrasonic energy is delivered “dry” and the system does include an applicator nozzle.

In certain embodiments, the generator includes a treatment algorithm that calculates an approximate treatment time. Alternatively, a physician or health professional can select the desired treatment time. For example, treatment time may be determined based on the area of the tissue for which treatment is desired. Thus, treatment time would be proportional to the area of the donor site or recipient site, as applicable. The area may optionally be calculated to include periwound tissue.

Generally, treatment times vary from approximately 3 minutes to approximately 25 minutes. However, shorter (approximately 1-3 minutes) and longer (25-30 minutes or greater than approximately 30 minutes) treatment times are contemplated. Once emitted energy, and fluid spray when applicable, emerge from the applicator, the operator can direct the energy to the selected treatment site. In one recommended embodiment, the treatment site is treated by slowly moving the applicator head back and forth and/or up and down (at a non-contact distance) across the site. The spray pattern may be, for example, serpentine or substantially checkerboard in pattern. This delivery method has two advantages. First, this method helps insure that ultrasonic energy and liquid spray are delivered to the entire treatment site. Second, this method may help prevent operator fatigue that would likely result if the device was held in substantially the same place throughout the treatment. In one embodiment, the applicator is held such that the ultrasonic energy and liquid spray are delivered substantially normal to the surface of the treatment site. Additionally, the spray pattern may include moving the applicator in-and-out relative to the wound surface (e.g., varying the distance from the wound while maintaining a non-contact distance). Such a spray pattern helps ensure that a treatment site is treated completely and at an effective distance.

In one embodiment, the need for a human operator is eliminated. The transducer assembly is affixed to a robotic arm programmed to direct the emitted energy and liquid spray to the treatment site.

As outlined above, in certain embodiments the emitted ultrasonic energy and fluid spray are applied to the treatment site for a treatment time proportional to the size of the treatment site. In one embodiment, the invention provides a treatment algorithm for selecting treatment time based on the size of the treatment site. The time for each treatment is selected based on the area of the treatment site. The area of the wound is calculated by measuring the length of the treatment site (at its greatest point) and the width of the wound (at its greatest point and perpendicular to the length). The length and width of the treatment site can be measured, for example, in centimeters. The area of the treatment site (in square centimeters) is calculated by multiplying the length times the width of the treatment site. The treatment time is proportional to the area of the treatment site.

The present invention provides methods for using ultrasonic energy to improve skin grafting. By way of further non-limiting example, commonly-owned U.S. Pat. No. 6,569,099, and application Ser. Nos. 11/473,934, 10/409,272, 10/815,384, and 12/006,739 disclose ultrasonic systems, devices and methods for treatment. The entire contents of each of the foregoing patents and patent application are incorporated herein by reference. Briefly, these patents and applications discloses devices, systems, and methods for delivering ultrasound energy, in the presence or absence of a liquid spray, via an applicator. The ultrasound energy and, when present the liquid spray, is delivered from a non-contact distance. Commonly-owned U.S. patent application Ser. Nos. 11/473,934 and 12/006,739, the entire contents of which are incorporated herein by reference, additionally provide several examples of removable applicator nozzles that can be used with an ultrasound therapy device. The disclosed devices and systems can be used to deliver ultrasonic energy.

### (iv) Skin Grafting Methods

Skin covers the entire external surface of the human body and performs numerous specialized functions. It serves as a protective barrier preventing internal tissues from exposure to trauma, radiation, temperature changes, and infection. Other important functions include thermoregulation and control of fluid balance.

Restoration of an intact barrier is of critical importance following wounding. Depending on the nature of the wound, the health status of the patient, and the types of medical intervention available, the wound may heal sufficiently (e.g., complete or near complete closure) without the need for a graft. However, grafting may be useful or necessary to accelerate wound healing and/or to achieve complete closure of wounds that fail to heal sufficiently in the absence of a graft. The benefits of skin grafting may include accelerated healing of burns and other wounds, reduction of scar contracture, improved cosmetics, reduction of fluid loss, and protection from bacterial infection.

The skin consists of two layers, the epidermis and dermis. The epidermis, the more external of the two layers, is a stratified squamous epithelium consisting primarily of keratinocytes in progressive stages of differentiation from deeper to more superficial layers. The epidermis has no blood vessels, thus it must receive nutrients by diffusion from the underlying dermis through the basement membrane, which separates the two skin layers.

The dermis is a more complex structure and is composed of two layers, the more superficial papillary dermis and the deeper reticular dermis. The papillary dermis is thinner, consisting of loose connective tissue containing capillaries, elastic fibers, reticular fibers, and some collagen. The reticular dermis consists of a thicker layer of dense connective tissue containing larger blood vessels, closely interlaced elastic fibers, and coarse, branching collagen fibers arranged in layers parallel to the surface. The reticular layer also contains fibroblasts, mast cells, nerve endings, lymphatics, and some epidermal appendages. Surrounding the components of the dermis is the gel-like ground substance composed of mucopolysaccharides (primarily hyaluronic acid), chondroitin sulfates, and glycoproteins.

Epidermal appendages are important as a source of epithelial cells that re-epithelialize when the overlying epithelium is removed or destroyed in patients with partial thickness burns, abrasions, or split-thickness skin graft harvesting. These intradermal epithelial structures, such as sebaceous glands, sweat glands, and hair follicles, are lined with epithelial cells with the potential for division and differentiation. They are found deep within the dermis and in the subcutaneous fat deep to the dermis. Without being bound be theory, one benefit of delivering ultrasound energy from a non-contact distance to tissue may be in promoting proliferation and/or differentiation of these underlying epithelial cells, thereby promoting re-epithelialization and promoting healing.

Skin transplanted from one location to another on the same individual is termed an autogenous graft, or autograft. These consist of the entire epidermis and a dermal component of variable thickness. If the entire thickness of the dermis is included, the appropriate term is full-thickness skin graft. If less than the entire thickness of the dermis is included, appropriate terms are partial or split-thickness skin graft. Split-thickness skin grafts are further categorized as thin (0.005-0.012 inches), intermediate (0.012-0.018 inches), or thick (0.018-0.030 inches) based on the thickness of graft harvested.

The thicker the dermal component, the more the characteristics of normal skin are maintained following grafting. This is because of the greater collagen content and the larger number of dermal vascular plexuses and epithelial appendages contained within thicker grafts. However, thicker grafts require more favorable conditions for survival because of the greater amount of tissue requiring revascularization. The choice between full- and split-thickness skin grafting depends on wound condition, location, and size as well as aesthetic concerns.

Full-thickness skin grafts are most appropriate for visible areas of the face. Full-thickness grafts retain more of the characteristics of normal skin including color, texture, and thickness when compared with split-thickness grafts. Full-thickness grafts also undergo less contraction while healing. This is important on the face as well as on the hands and over mobile joint surfaces. Full-thickness grafts in children are more likely to grow with the individual. However, full-thickness skin grafts are limited to relatively small, uncontaminated, well-vascularized wounds and thus do not have as wide a range of application as split-thickness grafts. Donor sites must be closed primarily or, more rarely, resurfaced with a split-thickness graft from another site.

Split-thickness skin grafts can tolerate less ideal conditions for survival and have a much broader range of application. They are used to resurface large wounds, line cavities, resurface mucosal deficits, close donor sites of flaps, and resurface muscle flaps. They also are used to achieve temporary closure of wounds created by the removal of lesions that require pathologic examination prior to definitive reconstruction. Split-thickness skin graft donor sites heal more readily, and these donor sites may even be re-harvested once healing is complete.

Split-thickness grafts are more fragile, especially when placed over areas with little underlying soft tissue bulk for support, and usually cannot withstand subsequent radiation therapy. They contract more during healing, do not grow with the individual, and tend to be smoother and shinier than normal skin because of the absence of skin appendages in the graft. They tend to be abnormally pigmented, either pale or white, or alternatively hyperpigmented, particularly in darker-skinned individuals. Their lack of thickness, abnormally smooth texture, lack of hair growth, and abnormal pigmentation make these grafts more functional than cosmetic. Finally, there is often pain at the donor site following graft harvest. One benefit of the subject methods is that the delivery of ultrasonic energy to the donor site can be used to decrease and/or relieve pain at the donor site.

Selection of the donor site is usually based on the features wanted at the recipient site. Additionally, the selection of the donor site, particularly when the graft is an autograft, depends on the health of the patient and the availability of healthy tissue for harvest. The selection of the donor site is more important in full-thickness grafts, because more of the characteristics of the donor site skin will be retained by the grafted material in its new location. Thickness, texture, pigmentation, and presence or absence of hair should be matched as closely as possible. When grafting in children, consider that donor sites such as the groin, axillae, thigh, and chest will grow hair at puberty, and this hair growth may be undesirable at the new location. Donor sites for full-thickness grafts also are chosen to be inconspicuous and easily closed primarily.

Full-thickness grafts may be harvested from the upper eyelid, nasolabial fold, pre- and postauricular regions, and the supraclavicular fossa. These donor sites most often are employed to close a wound on the face or neck. When harvesting from the face, it is often aesthetically preferable to harvest bilaterally to maintain facial symmetry, even if the result is more skin being removed than is necessary to cover the defect.

Split-thickness skin grafts may be harvested from any surface of the body, but sites should be chosen that are easily concealed in recreational clothing. Common sites include the upper anterior and lateral thigh. The buttocks may be used as a donor site, but the patient may complain of significant postoperative pain and will require assistance caring for the wound. The scalp is used for resurfacing areas of the face too large for a full-thickness graft and is especially useful in severe burns with limited donor site availability. Because of its thickness, scalp skin may be repeatedly harvested with almost no risk of alopecia or subsequent hair growth at the recipient site. For hand wounds, the upper inner arm is a cosmetically appealing donor site.

A skin graft is the removal and transplantation of healthy skin from one area of the body (source area or donor site) to another area (recipient area) where the skin has been damaged. The donor sites most commonly used for skin grafts are the inner thigh, leg, buttocks, upper arm, and forearm. However, the use of the term donor site is intended to refer to a graft harvested from any area of the patient's body.

As detailed above, there are three main types of skin graft techniques: split-thickness graft, full-thickness graft, and composite graft. In a split-thickness graft, the top layer of skin (epidermis) and part of the middle layer (dermis) of skin are removed from the donor site. This type of graft allows the donor site to heal more quickly. However, the graft is also more fragile, and may be abnormally pigmented. This may increase the healing time at the recipient site, result in more scarring or discoloration following grafting, and/or result in a higher incidence of graft failure (e.g., scenarios where the graft fails to “take”, in whole or in part, thus necessitating additional grafting or supplementation with synthetic tissue). Split-thickness grafting is the most common skin graft used.

In a full-thickness graft, an entire area of skin is removed from the donor site and transferred to the recipient site. Although this type of graft often requires stitches to facilitate closure and healing of the donor site, the final outcome at the recipient site is usually better. Full-thickness grafts are usually recommended for recipient site areas where cosmetic appearance is important, such as the face. However, full-thickness grafts can only be placed on areas of the body that have significant vascularization (blood vessels). This requirement somewhat limits the use of this type of graft.

In a composite graft, a combination of tissue (skin and fat; skin and cartilage; or dermis and fat), is used. This type of graft is sometimes used when the recipient site is an area that require three-dimensionality, such as the nose.

The present invention provides methods for improving skin grafting using ultrasound energy delivered at a non-contact distance. The invention contemplates delivering ultrasound energy from a non-contact distance to a donor site and/or a recipient site. The methods of the invention can be used, regardless of the type of grafting technique used to harvest the tissue from the donor site.

The use of one's own skin as the donor area is called an autograft. However, if there is not enough suitable skin on a patient's own body to provide graft coverage for another area on the same body, then skin may be harvested from outside sources. Exemplary alternative sources include an allograft obtained from a living human donor, an allograft obtained from a human cadaver, a xenograft from an animal source, and synthetic tissue. Typically, although not always, tissue harvested from one of these alternate sources is only meant to provide temporary protection until the patient's own tissue heals.

The present invention provides methods for improving skin grafting using ultrasound energy delivered at a non-contact distance. The invention contemplates delivering ultrasound energy from a non-contact distance to a donor site and/or a recipient site. Although the graft is preferably an autograft, and thus the donor site and recipient site are preferably in the same patient, the present methods can also be used when the graft is an allograft.

Skin grafts are used in numerous contexts to promote healing of wounds. For example, sometimes a large wound does not achieve complete closure, and a skin graft is used to facilitate closure. Additionally, chronic wounds, particularly wounds in patients with a significant underlying health condition that decreases wound healing capacity and/or vascular health, often heal very slowly and/or incompletely.

One of the benefits of the methods of the present invention is that ultrasound energy is delivered from a non-contact distance. Given that treatment is delivered without direct contact between the device and wound tissue, the present methods can be used on any area of the body, regardless of the cause of the underlying wound, and regardless of the patient's overall medical condition. By may of non-limiting example, the present methods can be used regardless of whether the underlying wound is due to a burn, traumatic injury, venous ulcer, arterial ulcer, diabetic ulcer, or pressure ulcer. By way of additional illustration, the present methods can be used when skin grafting is performed in the surgical context, for example, as part of a cosmetic procedure or medical reconstructive procedure (e.g., breast reconstruction following mastectomy).

Typically, some variation of the following general procedure is used when performing a skin graft. Prior to any procedure, the wound is cleaned. Depending on the nature of the wound, and the technique used to harvest tissue from the donor site, a suitable anesthesia is selected and administered. For example, the patient may be given a local, regional, or general anesthesia.

Prior to or following administration of anesthesia, the wound is measured. Following measurement of the wound, a pattern reflecting the amount of tissue required for the graft is made and outlined over a suitable donor site. The donor site is selected based on the amount of tissue needed, the technique of harvest (e.g., full-thickness, split-thickness, etc.), the health of the patient and the patient's other skin, etc. Once selected, the donor tissue is removed with a scalpel or dermatome. Depending on the size and nature of the harvested graft, the donor site may be closed with stitches, fastened with staples, sutures, or other closures, secured with a bandage, or simply cleaned and dressed.

The harvested graft is placed on the recipient site. The graft is typically fastened with stitches or staples—although a physician may readily choose the preferred methods for graft placement. Under certain circumstance, it may be desirable or necessary to mesh the graft prior to placing on the recipient site.

Following graft placement, the recipient site can be suitably dressed and managed using standard wound care practices. Optionally, a vacuum apparatus can be used to help control drainage. Within 36 hours following graft placement, new blood vessel should begin to grow to promote graft vascularization and epithelial cell proliferation.

The complications associated with standard grafting procedures include bleeding, graft failure, infection in either the donor or recipient site, scarring at either the donor or recipient site, changes in sensation at either the donor or recipient site, graft contraction, lack of graft adherence, and pain at either the donor or recipient site.

As detailed throughout, the present invention provides methods for delivering ultrasonic energy to a donor site and/or recipient site as part of an overall process for managing skin grafting. Delivering of ultrasonic energy can be combined with any medically appropriate procedures for selecting a donor site, preparing a donor site for harvest, harvesting a skin graft, transplanting a skin graft, caring for the donor site post-harvest, caring for the recipient site pre-harvest, and/or caring for the recipient site post-harvest.

Proper preparation of the recipient site is important for the success of the skin graft. Physiologic conditions must be optimized to accept and nourish the graft. Skin grafts do not thrive without sufficient blood supply. Skin grafts will survive on periosteum, perichondrium, peritenon, perineurium, dermis, fascia, muscle, and granulation tissue.

The wound at the recipient site should be relatively free from necrotic tissue and the presence of bacteria should be decreased or substantially controlled. Bacterial counts greater than 100,000 per square centimeter are associated with a high likelihood of graft failure. To achieve an adequate wound bed, debridement, dressing changes, and topical or systemic antibiotics may be indicated prior to grafting. In certain embodiments of the present invention, ultrasonic energy is used as part of the process of preparing the recipient site prior to skin graft transplantation.

Careful operative technique is important for graft survival. After administering appropriate local, regional, or general anesthesia, the wound is prepared for grafting. Preparation includes, but is not limited to cleansing the wound with saline or dilute Betadine and achievement of good hemostatic control.

Full-thickness skin grafts are typically harvested with a scalpel. The wound is measured, a pattern is made, and the pattern is outlined over the donor region. The pattern should be enlarged by 3-5% to compensate for the immediate primary contraction that occurs because of the elastic fibers contained in the graft dermis. The donor site then may be infiltrated with local anesthetic with or without epinephrine. Infiltration should be performed after the outline of the graft has been drawn on the skin to avoid distortion caused by the infiltrated volume. After incising the pattern, the skin is elevated with a skin hook keeping a finger of the nonoperating hand on the epidermal side of the graft. This provides tension and a sense of graft thickness while the operating hand dissects the graft off the underlying subcutaneous fat.

Any residual adipose tissue must be trimmed from the underside of the graft, because this fat is poorly vascularized and will prevent direct contact between the graft dermis and the wound bed. Trimming of residual subcutaneous fat is best accomplished with a sharp, curved scissor with the graft stretched over the nonoperating hand until only the white, glistening dermis remains on the deep surface.

Split-thickness grafts may be harvested in several ways. The most common technique involves use of a blade dermatome, which provides rapid harvest of large grafts of uniform thickness. Dermatomes may be air powered, electric, or manually operated. Commonly used dermatomes include the Castroviejo, Reese, Padgett-Hood, Brown, Davol-Simon, and Zimmer. All of these harvest by the same mechanism: a rapidly oscillating side-to-side blade is advanced over the skin with thickness and width settings adjusted by the surgeon. Regardless of the instrument used, adequate anesthesia must be established, because harvesting of skin grafts is a painful procedure. Lidocaine with epinephrine injected at the donor site may reduce blood loss and provide greater tissue turgor, which assists in harvesting.

A second method for harvesting split-thickness grafts is with a drum dermatome. Drum dermatomes (Reese, Padgett-Hood) are less frequently used today but are available for specialized grafting needs. On these instruments, the oscillating blade is manually powered as the drum is rolled over the skin surface. These dermatomes can be used to harvest broad sheets of skin of exacting thickness. They are useful when the donor site is irregular, with a convexity, concavity, or bony prominence (neck, flank, buttock), because the skin to be harvested is first made adherent to the drum with a special glue or adhesive tape. These dermatomes also allow precise irregular patterns to be harvested by varying the pattern of adhesive applied to the skin and drum.

Another method for harvesting split-thickness grafts is free-hand with a knife. Although this may be performed with a scalpel, other devices such as the Humby knife, Weck blade, and Blair knife also are available.

Regardless of the technique used to harvest the graft, Betadine or another solution may be used to sterilize the donor site at the beginning of the procedure. It may be useful to lubricate the skin and dermatome with mineral oil or pHisoHex to facilitate easy gliding of the dermatome over the skin. These may be gently washed from the skin graft with saline following harvesting but do not compromise graft survival.

The dermatome is held in the dominant hand of the operator at a 30-45° angle from the donor skin surface. Greater angulation of the dermatome leads to gouging or trenching of the donor site skin. With the nonoperating hand providing traction behind the dermatome and the assistant providing traction in front of the dermatome, the dermatome is activated and advanced in a smooth, continuous motion over the skin with gentle downward pressure. After the appropriate length has been harvested, the dermatome is tilted away from the skin and lifted off the skin to cut the distal edge of the graft and complete the harvesting. The graft then may be gently washed to remove the lubricant and wrapped in a moistened saline sponge until it is ready to be used.

Once harvested, a split-thickness skin graft may optionally be meshed by placing the graft on a carrier and passing it through a mechanical meshing instrument. This technique allows expansion of the surface area of the graft up to nine times the surface area of the donor site. This technique is useful when insufficient donor skin is available for a large wound, such as in major burns or when the recipient site is irregularly contoured. The slits in the meshed skin graft allow wound fluid to escape through the graft rather than accumulating beneath it and preventing adherence.

When healed, the grafted site characteristically has a “crocodile skin” or “checkerboard” appearance. Because of the secondary contraction and poor cosmesis with this technique, it should be avoided over joints and in the face, hands, and other highly visible areas.

Once the graft has been harvested, the recipient site should be re-inspected for hemostasis. Once this is complete, the graft may be placed on the wound bed. Attention must be paid to placing the dermal side down. The graft must then be secured in place to provide stability during initial adherence and healing. This is most often accomplished by suturing or stapling the graft to the skin surrounding the wound bed.

In the days immediately following skin graft transplantation to the recipient site, the skin graft is fragile and needs to “take”. As such, it may be preferable to suspend or delay ultrasonic therapy at the recipient site, as disclosed herein, until the graft has had a chance to “take”. A treatment professional can assess the progress of the graft and determine when ultrasonic therapy at the recipient site may begin or resume. For example, it is often preferable to suspend or delay ultrasonic therapy at the recipient site for approximately seven, eight, nine, or ten days following skin graft transplantation. Sometimes, it may even be preferable to delay treatment for more than ten days or even approximately two weeks or more. Note that suspension of treatment of the recipient site does not influence treatment of the donor site. The donor site may be treated with ultrasonic energy at any point prior to or following removal of the skin graft.

As detailed throughout, the present invention provides numerous methods for using ultrasonic energy to prepare and/or manage the donor site and/or recipient site during one or more phases of skin grafting. The exemplary methods of medically acceptable techniques and procedures used to prepare the donor site and recipient, to harvest a graft at the donor site, and to transplant a graft to the recipient site are merely exemplary.

### (v) Other Wound Treatment Modalities

The methods of the present invention can be used alone or in combination with one or more existing methods for managing wounds and/or skin grafts. By way of example, certain available wound care agents and methods are described briefly below. Any one or more of these additional treatment modalities can be used in combination with ultrasound therapy.

Dressings

Dressings are often used to keep a wound clean, to serve as a carrier for topical medicaments, and/or to promote healing. Similarly, dressings are often used in the context of skin grafts. Depending on the size and type of skin graft, dressings and/or topical medicaments may be used at both the recipient site and the donor site.

In the case of dressings for use in the skin graft context, a dressing is typically chosen to provide uniform pressure over the entire grafted area with a nonadherent, semiocclusive, absorbent dressing material. These dressings are intended to immobilize the graft, prevent shearing, and prevent seroma or hematoma formation beneath the graft. Note, however, that although the recipient site is often dressed, the graft may be treated open with no dressing and with a layer of ointment to prevent desiccation.

Topical Medicaments

Topical medicines may be applied directly to the tissue or applied via a dressing. For example, silver is often delivered via a silver-based dressing. Additional medicaments include anti-septics, anti-bacterial agents, topical pain relieving agents, and anti-fungal agents. Topical medicaments may be applied prior to, concurrently with, or following delivery of ultrasonic energy. Moreover, topical medicaments may be used during all or only a portion of a patient's treatment. Combinations of topical medicaments, applied at the same or differing times, are also contemplated.

Other exemplary modalities include Negative Pressure Therapy or Vacuum Assisted Closure (VAC) and hyperbaric therapy.

The ultrasonic methods described herein can be used alone or in combination with one or more additional treatment modalities as part of a therapeutic regimen to care for patients requiring a skin graft. Dressings, topical medicaments, hyperbaric therapy, and negative pressure therapy are exemplary of other modalities that can be used.

Systemic Medicaments

Some patients may be treated with systemic (oral or intravenous) antibiotics, anti-viral agents, or anti-fungal agents. These systemic treatments may be administered prophylactically if the patient is deemed to be at high risk for infection or if the patient's overall condition suggests that an infection could be dangerous or life threatening. Alternatively, systemic treatments may be administered following some evidence or symptoms of infection. The ultrasound methods provided herein can be delivered in combination with systemic medicaments. Systemic medicaments or combination of systemic medicaments can be administered on any medically acceptable time table.

Additionally, some patients may be placed on immunosuppressive therapy. For example, a patient receiving an allograft (rather than an autograft) may be treated with medications to reduce the risk of rejecting the autograft. This is particularly true if the autograft is from a source other than an identical twin. The ultrasound methods provided herein can be delivered in combination with anti-rejection or other immunosuppressive therapy. Immunosuppresive therapies or combinations of immunosuppresive therapies can be administered on any medically acceptable time table.

### EXEMPLIFICATIONS

The invention now being generally described, it will be more readily understood by reference to the following examples, which are included merely for purposes of illustration of certain aspects and embodiments of the present invention, and are not intended to limit the invention.

### Example 1

### Preparing a Donor Site

A suitable donor site is selected. Prior to harvesting the skin graft from the donor site, the required size and shape of the graft will be determined to insure that an appropriate skin graft is harvested. However, a decision regarding the exact size and shape of the skin graft is not needed in order to prepare the donor site.

Without being bound by theory, one or more pre-treatments of the donor site with ultrasonic energy (wet or dry) may be used. The treatment area is approximately equal to the area of the anticipated skin graft. Optionally, periwound tissue (tissue that will not be removed but will be adjacent to the wound created at the donor site following harvest of the skin graft) is also treated.

A system for delivering low frequency ultrasonic energy is provided. An operator uses the system to deliver to the donor site low frequency ultrasonic energy. An exemplary system, and instructions for using the system, is available from Celleration, Inc. and sold as the MIST Therapy® System. Although the ultrasonic energy may be delivered “wet” or “dry”, in this example the ultrasonic energy is delivered “wet” with a saline spray. Thus, the operator delivers low frequency ultrasonic energy and a saline spray to the treated patient tissue.

The energy is delivered to the donor site from a non-contact distance between the vibrating ultrasound transducer tip and the treated donor site tissue. In this example, the vibrating transducer tip is shielded with an applicator nozzle that also serves to direct the liquid to the vibrating transducer and, ultimately to the patient tissue. The energy is delivered to the donor site from a non-contact distance and without direct contact between the treated donor site tissue and either the vibrating transducer tip or the applicator nozzle.

The operator delivers the ultrasonic energy and the saline spray to the donor site. The total treatment time may vary according to the health care professional's recommendation. Exemplary treatment time is proportional to the area of the donor site and, optionally is proportional to the area of the donor site+the periwound region.

Once the system is turned on (the transducer is vibrating and liquid is flowing to the transducer) the operator delivers the ultrasonic energy and the saline spray. To provide treatment to the entire treatment area, the operator may deliver the ultrasonic energy and the saline spray by slowly moving the applicator head back and forth and/or up and down (at a non-contact distance) across the area of the donor site. The spray pattern may be, for example, serpentine or substantially checkerboard in pattern.

Preparation of the donor site may include a single treatment of the donor site prior to harvest of the skin graft. Alternatively, preparation of the donor site may include multiple treatments prior to harvest of the skin graft. Further, the last pre-harvest treatment may occur essentially just prior to harvest or it may occur hours (e.g., about 0.5, 1, 1.5, 2, 3, 4, 5, 6, 8 hours), days, or even weeks prior to harvest. When the last pre-harvest treatment occurs essentially just prior to harvest, such a treatment may optionally occur in the operating room as part of an initial step in the surgical procedure to harvest the skin graft. However, pre-harvest treatment in the patient's room or in another non-surgical room is similarly contemplated.

### Example 2

### Donor Site Management

Regardless of whether ultrasonic energy is used to prepare a donor site prior to skin graft removal, ultrasonic energy can be used to manage the donor site following removal of the skin graft. In other words, ultrasonic energy can be used to manage the healing of the donor site following removal of the skin graft.

Without being bound by theory, the donor site can be treated with ultrasonic energy (wet or dry) one or more times following skin graft harvest. The treatment area is approximately equal to the area of skin graft. In other words, the treatment area is approximately equal to the wound created at the donor site following harvest of the skin graft. Optionally, periwound tissue adjacent to the wound created at the donor site following harvest of the skin graft is also treated.

A system for delivering low frequency ultrasonic energy is provided. An operator uses the system to deliver to the donor site low frequency ultrasonic energy. An exemplary system, and instructions for using the system, is available from Celleration, Inc. and sold as the MIST Therapy® System. Although the ultrasonic energy may be delivered “wet” or “dry”, in this example the ultrasonic energy is delivered “wet” with a saline spray. Thus, the operator delivers low frequency ultrasonic energy and a saline spray to the treated patient tissue.

The energy is delivered to the donor site from a non-contact distance between the vibrating ultrasound transducer tip and the treated donor site tissue. In this example, the vibrating transducer tip is shielded with an applicator nozzle that also serves to direct the liquid to the vibrating transducer and, ultimately to the patient tissue. The energy is delivered to the donor site from a non-contact distance and without direct contact between the treated donor site tissue and either the vibrating transducer tip or the applicator nozzle.

The operator delivers the ultrasonic energy and the saline spray to the donor site. The total treatment time may vary according to the health care professional's recommendation. Exemplary treatment time is proportional to the area of the donor site and, optionally is proportional to the area of the donor site+the periwound region.

Once the system is turned on (the transducer is vibrating and liquid is flowing to the transducer) the operator delivers the ultrasonic energy and the saline spray. For example, to help deliver ultrasonic energy and saline spray to the entire treatment area, the operator may deliver ultrasonic energy and saline spray by slowly moving the applicator head back and forth and/or up and down (at a non-contact distance) across the area of the donor site. The spray pattern may be, for example, serpentine or substantially checkerboard in pattern.

The donor site may be treated a single time (one treatment) post-skin graft removal or multiple times. For example, the donor site may be treated approximately twice per week for at least 1, 2, 3, 4, 5, or 6 weeks (multiple treatments).

Depending on the size and type of skin graft (e.g., full-thickness, partial thickness), donor site management may also include one or more other treatment modalities. For example, the donor site may be covered with a dressing or treated with a topical antibiotic ointment or other medicament. Additionally or alternatively, the patient may be given oral or intravenous antibiotics.

### Example 3

### Reducing Pain Following Harvest from Donor Site

Regardless of whether ultrasonic energy is used to prepare a donor site prior to skin graft removal, ultrasonic energy can be used to reduce pain associated with graft harvest, as well as the pain experienced following removal of the skin graft from the donor site.

Without being bound by theory, the donor site can be treated with ultrasonic energy (wet or dry) one or more times prior to and/or following skin graft harvest. The treatment area is approximately equal to the area of skin graft. In other words, the treatment area is approximately equal to the wound created at the donor site following harvest of the skin graft. Optionally, periwound tissue adjacent to the wound created at the donor site following harvest of the skin graft is also treated.

A system for delivering low frequency ultrasonic energy is provided. An operator uses the system to deliver to the donor site low frequency ultrasonic energy. An exemplary system, and instructions for using the system, is available from Celleration, Inc. and sold as the MIST Therapy® System. Although the ultrasonic energy may be delivered “wet” or “dry”, in this example the ultrasonic energy is delivered “wet” with a saline spray. Thus, the operator delivers low frequency ultrasonic energy and a saline spray to the treated patient tissue.

The energy is delivered to the donor site from a non-contact distance between the vibrating ultrasound transducer tip and the treated donor site tissue. In this example, the vibrating transducer tip is shielded with an applicator nozzle that also serves to direct the liquid to the vibrating transducer and, ultimately to direct the liquid spray to the patient tissue. The energy is delivered to the donor site from a non-contact distance and without direct contact between the treated donor site tissue and either the vibrating transducer tip or the applicator nozzle.

The operator delivers the ultrasonic energy and the saline spray to the donor site. The total treatment time may vary according to the health care professional's recommendation. Exemplary treatment time is proportional to the area of the donor site and, optionally is proportional to the area of the donor site+the periwound region.

Once the system is turned on (the transducer is vibrating and liquid is flowing to the transducer) the operator delivers the ultrasonic energy and the saline spray. For example, to help deliver ultrasonic energy and saline spray to the entire treatment area, the operator may deliver ultrasonic energy and saline spray by slowly moving the applicator head back and forth and/or up and down (at a non-contact distance) across the area of the donor site. The spray pattern may be, for example, serpentine or substantially checkerboard in pattern.

The donor site may be treated a single time (one treatment) post-skin graft removal or multiple times. For example, the donor site may be treated approximately twice per week for at least 1, 2, 3, 4, 5, or 6 weeks (multiple treatments). Optionally, the donor site may be treated one or more times prior to skin graft removal.

Reduction in pain can be evaluated based on patient self-reporting. Reduction in pain can also be evaluated based on the patient's request for or reliance on pain medication relative to, for example, the level of pain medication typically required by similar patients whose treatment does not include ultrasound therapy. Reduction in pain can also be evaluated based on the level of pain experienced by the patient at a time just prior to the next scheduled treatment (e.g., the longest duration between treatments) versus the level of pain experienced at a time more proximal to treatment.

Depending on the size and type of skin graft (e.g., full-thickness, partial thickness), pain management and/or pain reduction may also include one or more other treatment modalities. For example, the donor site may be covered with a dressing or treated with a topical antibiotic ointment, analgesic, or other medicament. Additionally or alternatively, the patient may be given oral or intravenous antibiotics, analgesics, anti-inflammatories, narcotics, or other pain management medications.

It is to be understood that the foregoing description is merely a disclosure of particular embodiments and is in no way intended to limit the scope of the disclosure. All operative combinations of any of the foregoing aspects and embodiments are contemplated and are within the scope of the invention. Other possible modifications will be apparent to those skilled in the art and all modifications will be apparent to those in the art and all modifications are to be defined by the following claims.

### INCORPORATION BY REFERENCE

All publications and patents mentioned herein, are hereby incorporated by reference in their entirety as if each individual publication or patent was specifically and individually indicated to be incorporated by reference.

1-14. (canceled)

15. A method of preparing tissue for skin grafting, comprising
providing a transducer which can emit low frequency ultrasonic energy;
delivering said ultrasonic energy to a skin graft donor site;
wherein said ultrasonic energy is delivered from a non-contact distance between a vibrating tip of the transducer and treated patient tissue, and wherein the delivered ultrasonic energy provides a therapeutic effect.

16. The method of claim 15, wherein the ultrasonic energy is delivered prior to removing a skin graft from said donor site, following removing a skin graft from said site, or both prior to and following removing a skin graft from said site.

17. (canceled)

18. (canceled)

19. The method of claim 15, wherein the ultrasonic energy is delivered via a liquid spray, and the method comprises delivering the low frequency ultrasonic energy and the liquid spray to said skin graft donor site and/or skin graft recipient site.

20. The method of claim 19, wherein the liquid spray is generated by delivering liquid to a distal portion of the transducer.

21. The method of claim 15, wherein the low frequency ultrasonic energy is delivered in the absence of a liquid spray or coupling agent.

22. The method of claim 15, wherein said method further comprises delivering ultrasonic energy to a skin graft recipient site.

23. The method of claim 15, wherein the method further comprises
harvesting a skin graft from said donor site; and
transplanting said skin graft to a recipient site.

24. The method of claim 23, wherein the skin graft is a full-thickness skin graft or a partial thickness skin graft.

25. The method of claim 16, wherein the skin graft is an autograft or an allograft.

26. (canceled)

27. The method of claim 15, wherein the method is part of a therapeutic regimen combining one or more additional treatment modalities.

28. The method of claim 27, wherein the one or more additional treatment modalities comprises applying a topical medicament to the skin graft donor site and/or skin graft recipient site prior to and/or following delivering said ultrasonic energy to said skin graft donor site.

29. (canceled)

30. The method of claim 15, wherein the method comprises delivering said ultrasonic energy to both the skin graft donor site and a skin graft recipient site, and wherein said ultrasonic energy is delivered simultaneously or at differing times.

31. The method of claim 30, wherein the skin graft donor site and skin graft recipient site are on the same patient.

32. The method of claim 30, wherein the skin graft donor site and skin graft recipient site are on different individuals.

33. The method of claim 15, wherein the therapeutic effect is selected from one or more of: decreasing bacteria at the donor site and/or the recipient site; promoting wound healing of a wound produced at the donor site; promoting wound healing of the recipient site; promoting epithelial cell proliferation at the donor site following graft removal; promoting epithelial cell proliferation at the recipient site following graft transplantation; promoting graft survival; decreasing inflammation at the donor site and/or recipient site; decreasing pain at the donor site and/or recipient site; decreasing or preventing scarring at the donor site and/or recipient site; decreasing or preventing biofilm formation; reducing pain associated with removal and/or receipt of a skin graft; and decreasing the risk of infection.

34. The method of claim 33, wherein the delivered ultrasonic energy penetrates treated patient tissue to provide said therapeutic effect.

35. The method of claim 33, wherein the delivered ultrasonic energy provides said therapeutic effect at the surface of the donor site and/or recipient site.

36. A method of preparing a skin graft donor site, comprising:
providing a transducer which can emit low frequency ultrasonic energy;
delivering said ultrasonic energy to a skin graft donor site at least one of prior to or following removing a skin graft from said site;
wherein said ultrasonic energy is delivered from a non-contact distance between a vibrating tip of the transducer and the skin graft donor site, and wherein the delivered ultrasonic energy provides a therapeutic effect.

37. The method of claim 36, wherein said ultrasonic energy is delivered to said skin graft donor site and to a periwound area adjacent to said skin graft donor site.

38. The method of claim 36, wherein said ultrasonic energy is delivered for a time proportional to one of an area of the skin graft donor site or an area of the skin graft donor site plus a periwound area.

39. (canceled)

40. The method of claim 36, wherein the method further comprises:
harvesting a skin graft from said donor site; and
transplanting said skin graft to a recipient site.

41. The method of claim 40, wherein the skin graft is a full-thickness skin graft or a partial-thickness skin graft.

42. (canceled)

43. The method of claim 40, wherein the skin graft is an autograft or an allograft.

44-46. (canceled)

47. The method of claim 40, wherein the method further comprises delivering ultrasonic energy to the recipient site following transplantation of said skin graft to said recipient site, wherein said ultrasonic energy is delivered from a non-contact distance between a vibrating tip of the transducer and the skin graft recipient site, and wherein the delivered ultrasonic energy provides a therapeutic effect.

48. The method of claim 36, wherein the ultrasonic energy is delivered via a liquid spray, and the method comprises delivering the low frequency ultrasonic energy and the liquid spray to said skin graft donor site.

49. The method of claim 48, wherein the liquid spray is generated by delivering liquid to a distal portion of the transducer.

50. The method of claim 36, wherein the low frequency ultrasonic energy is delivered in the absence of a liquid spray or coupling agent.

51. The method of claim 36, wherein the method is part of a therapeutic regimen combining one or more additional treatment modalities.

52. The method of claim 51, wherein the one or more additional treatment modalities comprises applying a topical medicament to the skin graft donor site prior to and/or following delivering said ultrasonic energy to said skin graft donor site.

53. The method of claim 36, wherein the method comprises at least one treatment multiple treatments of ultrasonic energy prior to removing the skin graft from the skin graft donor site.

54. (canceled)

55. The method of claim 36, wherein the method comprises multiple treatments of ultrasonic energy following removing the skin graft from the skin graft donor site.

56. The method of claim 55, wherein the method comprises delivering ultrasonic energy at least twice per week for at least two weeks following removing the skin graft from the skin graft donor site.

57. The method of claim 36, wherein the therapeutic effect is selected from one or more of: decreasing bacteria at the donor site; promoting wound healing of a wound produced at the donor site; promoting epithelial cell proliferation at the donor site following graft removal; decreasing or preventing scarring at the donor site; decreasing inflammation at the donor site; decreasing pain at the donor site; decreasing or preventing biofilm formation; and decreasing the risk of infection.

58. The method of claim 47, wherein the therapeutic effect is selected from one or more of: decreasing bacteria at the donor site and/or the recipient site; promoting wound healing of a wound produced at the donor site; promoting wound healing of the recipient site; promoting epithelial cell proliferation at the donor site following graft removal; promoting epithelial cell proliferation at the recipient site following graft transplantation; promoting graft survival; decreasing inflammation at the donor site and/or recipient site; decreasing pain at the donor site and/or recipient site; decreasing or preventing scarring at the donor site and/or recipient site; decreasing or preventing biofilm formation; and decreasing the risk of infection.

59. The method of claim 57, wherein the delivered ultrasonic energy penetrates treated patient tissue to provide said therapeutic effect.

60. The method of claim 57, wherein the delivered ultrasonic energy provides said therapeutic effect at the surface of the donor site and/or recipient site.

61-76. (canceled)

77. The method of claim 15, wherein the ultrasonic energy is delivered at a frequency of approximately 20 kHz to approximately 200 kHz.

78. The method of claim 15, wherein the ultrasonic energy is delivered at a frequency of approximately 30 kHz to approximately 50 kHz.

79. The method of claim 15, wherein the ultrasonic energy is delivered at a frequency of approximately 40 kHz.

80. The method of claim 15, wherein the ultrasonic energy level provided to patient tissue is approximately 0.1 watts/cm²-1.0 watts/cm².

81. The method of claim 15, wherein the ultrasonic energy level provided to patient tissue is approximately 0.1 watts/cm²-0.7 watts/cm².

82. The method of claim 15, wherein the delivered ultrasonic energy penetrates patient tissue to a depth of at least about 2 millimeters.

83. The method of claim 15, wherein the delivered ultrasonic energy penetrates patient tissue to a depth of at least about 4 millimeters.

84. The method of claim 15, wherein the delivered ultrasonic energy decreases the healing time of a wound at the recipient site and/or a wound at the donor site.

85. The method of claim 19, wherein the liquid spray is selected from a saline solution or other substantially inert liquid.

86. The method of claim 19, wherein the liquid spray includes a therapeutic medicament.

87. The method of claim 15, wherein the ultrasonic energy is delivered without direct contact with treated patient tissue.
